(19) United States
(12) Patent Application Publication
(10) Pub. No.: US 2005/0080054 A1
(43) Pub. Date: Apr. 14, 2005

(54) Treating androgen decline in aging male (ADAM)-associated conditions with SARMS

(76) Inventors: James T. Dalton, Upper Arlington, OH (US); Duane D. Miller, Germantown, TN (US); Mitchell S. Steiner, Germantown, TN (US); Karen A. Veverka, Cordova, TN (US)

Correspondence Address: EITAN, PEARL, LATZER & COHEN ZEDEK LLP, 10 ROCKEFELLER PLAZA, SUITE 1001, NEW YORK, NY, 10020, US

(21) Appl. No.: 10/683,161

(22) Filed: Oct. 14, 2003

Related U.S. Application Data
(60) Provisional application No. 60/418,336, filed on Oct. 16, 2002.

(51) Int. Cl. A61K 31/66; A61K 31/32; A61K 31/21; A61K 31/165; A61K 31/195; A61K 31/277

(52) U.S. Cl. 514/114; 514/563; 514/493; 514/522; 514/514; 514/616; 514/618; 514/620; 514/621

(57) ABSTRACT

The present invention provides a method of treating, preventing, suppressing, inhibiting or reducing the incidence of an Androgen Decline in Aging Male (ADAM)-associated condition in a male subject, by administering to the subject a selective androgen receptor modulator (SARM) compound and/or its analog, derivative, isomer, metabolite, pharmaceutically acceptable salt, pharmaceutical product, hydrate, N-oxide, prodrug, polymorph, crystal, or any combination thereof. The present invention further provides a method of treating, preventing, suppressing, inhibiting or reducing the incidence of sexual dysfunction, decreased sexual libido, erectile dysfunction, hypogonadism, sarcopenia, osteopenia, osteoporosis, alterations in cognition and mood, depression, anemia, hair loss, obesity, benign prostate hyperplasia or prostate cancer due to ADAM in a male subject, by administering to the subject a selective androgen receptor modulator (SARM) compound and/or its analog, derivative, isomer, metabolite, pharmaceutically acceptable salt, pharmaceutical product, hydrate, N-oxide, prodrug, polymorph, crystal, or any combination thereof.

### CROSS REFERENCE TO RELATED APPLICATIONS

This application claims the benefit of U.S. Provisional Application Ser. No. 60/418,336, filed Oct. 16, 2002, which is incorporated in its entirety by reference herein,

### FIELD OF INVENTION

This invention generally relates to the prevention and treatment of Androgen Decline in Aging Male (ADAM)-associated conditions in a subject. More particularly, this invention relates to a method of treating, preventing, suppressing, inhibiting, or reducing an ADAM-associated condition in a male subject, for example sexual dysfunction, decreased sexual libido, erectile dysfunction, hypogonadism, sarcopenia, osteopenia, osteoporosis, alterations in cognition and mood, depression, anemia, hair loss, obesity, benign prostate hyperplasia and/or prostate cancer, by administering to the subject a selective androgen receptor modulator (SARM) compound and/or its analog, derivative, isomer, metabolite, pharmaceutically acceptable salt, pharmaceutical product, hydrate, N-oxide, prodrug, polymorph, crystal, or any combination thereof.

### BACKGROUND OF THE INVENTION

Androgen decline in the aging male (ADAM) refers to a progressive decrease in androgen production, common in males after middle age. The syndrome is characterized by alterations in the physical and intellectual domains that correlate with and can be corrected by manipulation of the androgen milieu.

ADAM is characterized biochemically by a decrease not only in serum androgen, but also in other hormones, such as growth hormone, melatonin and dehydroepiandrosterone. Clinical manifestations include fatigue, depression, decreased libido, sexual dysfunction, erectile dysfunction, sarcopenia, osteopenia, osteoporosis, benign prostate hyperplasia, hypogonadism, alterations in mood and cognition, depression, anemia, obesity, hair loss and prostate cancer.

The onset of ADAM is unpredictable and its manifestations are subtle and variable, which has led to a paucity of interest in its diagnosis, monitoring and treatment. Innovative approaches are urgently needed at both the basic science and clinical levels to treat ADAM. The present invention is directed to satisfying this need.

### SUMMARY OF THE INVENTION

The present invention provides a method of treating, preventing, suppressing, inhibiting or reducing the incidence of an Androgen Decline in Aging Male (ADAM)-associated condition in a male subject, by administering to the subject a selective androgen receptor modulator (SARM) compound and/or its analog, derivative, isomer, metabolite, pharmaceutically acceptable salt, pharmaceutical product, hydrate, N-oxide, prodrug, polymorph, crystal, or any combination thereof. The present invention further provides a method of treating, preventing, suppressing, inhibiting or reducing the incidence of sexual dysfunction, decreased sexual libido, erectile dysfunction, hypogonadism, sarcopenia, osteopenia, osteoporosis, alterations in cognition and mood, depression, anemia, hair loss, obesity, benign prostate hyperplasia or prostate cancer due to ADAM in a male subject, by administering to the subject a selective androgen receptor modulator (SARM) compound and/or its analog, derivative, isomer, metabolite, pharmaceutically acceptable salt, pharmaceutical product, hydrate, N-oxide, prodrug, polymorph, crystal, or any combination thereof.

In one embodiment, this invention relates to a method of treating a male subject suffering from an Androgen Decline in Aging Male (ADAM)-associated condition, comprising the step of administering to the subject a selective androgen receptor modulator (SARM) compound. In another embodiment, the method comprises administering an analog, derivative, isomer, metabolite, pharmaceutically acceptable salt, pharmaceutical product, hydrate or N-oxide, prodrug, polymorph or crystal of the SARM compound, or any combination thereof. In one embodiment, the male subject is an aging male subject.

In another embodiment, the present invention provides a method of preventing, suppressing, inhibiting or reducing the incidence of an ADAM-associated condition in a male subject, comprising the step of administering to the subject a selective androgen receptor modulator (SARM) compound. In another embodiment, the method comprises administering an analog, derivative, isomer, metabolite, pharmaceutically acceptable salt, pharmaceutical product, hydrate or N-oxide, prodrug, polymorph or crystal of the SARM compound, or any combination thereof. In one embodiment, the male subject is an aging male subject.

In another embodiment, the present invention provides a method of treating a male subject suffering from sexual dysfunction, decreased sexual libido, erectile dysfunction, hypogonadism, sarcopenia, osteopenia, osteoporosis, alterations in cognition and mood, depression, anemia, hair loss, obesity benign prostate hyperplasia and/or prostate cancer due to Androgen Decline in an Aging Male (ADAM), comprising the step of administering to the subject a selective androgen receptor modulator (SARM) compound. In another embodiment, the method comprises administering an analog, derivative, isomer, metabolite, pharmaceutically acceptable salt, pharmaceutical product, hydrate or N-oxide, prodrug, polymorph or crystal of the SARM compound, or any combination thereof. In one embodiment, the male subject is an aging male subject.

In another embodiment, the present invention provides a method of preventing, suppressing, inhibiting or reducing the incidence of an ADAM-associated condition selected from sexual dysfunction, decreased sexual libido, erectile dysfunction, hypogonadism, sarcopenia, osteopenia, osteoporosis, alterations in cognition and mood, depression, anemia, hair loss, obesity, benign prostate hyperplasia and/or prostate cancer in a male subject, comprising the step of administering to the subject a selective androgen receptor modulator (SARM) compound. In another embodiment, the method comprises administering an analog, derivative, isomer, metabolite, pharmaceutically acceptable salt, pharmaceutical product, hydrate or N-oxide, prodrug, polymorph or crystal of the SARM compound, or any combination thereof. In one embodiment, the male subject is an aging male subject.

In one embodiment, the SARM compound that is effective at treating, preventing, suppressing, inhibiting or reducing the incidence of the ADAM-associated condition is a compound represented by the structure of formula I: 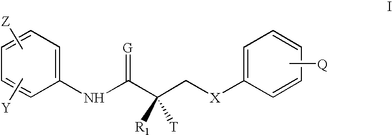
wherein
   G is O or S;
   X is a bond, O, CH₂, NH, Se, PR, NO or NR;
   T is OH, OR, —NHCOCH₃, or NHCOR
   Z is NO₂, CN, COOH, COR, NHCOR or CONHR;
   Y is CF₃, F, I, Br, Cl, CN, CR₃ or SnR₃;
   Q is alkyl, F, Cl, Br, I, CF₃, CN CR₃, SnR₃, NR₂, NHCOCH₃, NHCOCF₃, NHCOR, NHCONHR, NHCOOR, OCONHR, CONHR, NHCSCH₃, NHCSCF₃, NHCSR NHSO₂CH₃, NHSO₂R, OR, COR, OCOR, OSO₂R, SO₂R, SR, NCS, SCN, NCO, OCN; or Q together with the benzene ring to which it is attached is a fused ring system represented by structure A, B or C: 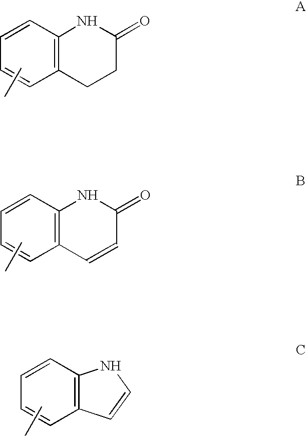
   R is alkyl, haloalkyl, dihaloalkyl, trihaloalkyl, CH₂F, CHF₂, CF₃, CF₂CF₃, aryl, phenyl, F, Cl, Br, I, alkenyl or OH; and
   R₁ is CH₃, CH₂F, CHF₂, CF₃, CH₂CH₃, or CF₂CF₃;
or its analog, derivative, isomer, metabolite, pharmaceutically acceptable salt, pharmaceutical product, hydrate or N-oxide, prodrug, polymorph or crystal of the SARM compound, or any combination thereof.

In another embodiment, the SARM compound that is effective at treating, preventing, suppressing, inhibiting or reducing the incidence of the ADAM-associated condition is a compound represented by the structure of formula II: 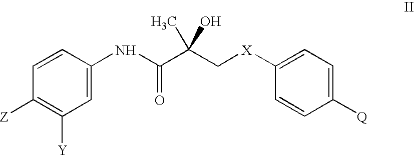
wherein
   X is a bond, O, CH₂, NH, Se, PR, NO or NR;
   Z is NO₂, CN, COOH, COR, NHCOR or CONHR;
   Y is CF₃, F, I, Br, Cl, CN, CR₃ or SnR₃;
   Q is alkyl, F, Cl, Br, I, CF₃, CN CR₃, SnR₃, NR₂, NHCOCH₃, NHCOCF₃, NHCOR, NHCONHR, NHCOOR, OCONHR, CONHR, NHCSCH₃, NHCSCF₃, NHCSR NHSO₂CH₃, NHSO₂R, OR, COR, OCOR, OSO₂R, SO₂R, SR, NCS, SCN, NCO, OCN; or Q together with the benzene ring to which it is attached is a fused ring system represented by structure A, B or C: 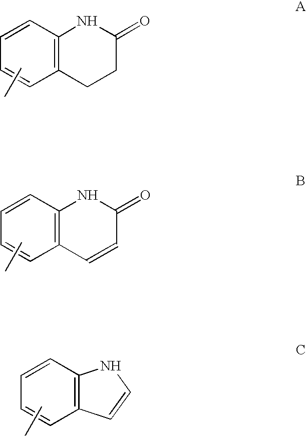
   R is alkyl, haloalkyl, dihaloalkyl, trihaloalkyl, CH₂F, CHF₂, CF₃, CF₂CF₃, aryl, phenyl, F, Cl, Br, I, alkenyl or OH;
or its analog, derivative, isomer, metabolite, pharmaceutically acceptable salt, pharmaceutical product, hydrate or N-oxide, prodrug, polymorph or crystal of the SARM compound, or any combination thereof.

In another embodiment, the SARM compound that is effective at treating, preventing, suppressing, inhibiting or reducing the incidence of the ADAM-associated condition is a compound represented by the structure of formula III: 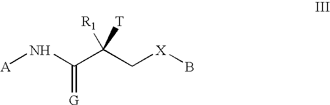
wherein
   X is a bond, O, CH₂, NH, Se, PR, NO or NR;
   G is O or S;
   R₁ is CH₃, CH₂F, CHF₂, CF₃, CH₂CH₃, or CF₂CF₃;
   T is OH, OR, —NHCOCH₃, or NHCOR;
   R is alkyl, haloalkyl, dihaloalkyl, trihaloalkyl, CH₂F, CHF₂, CF₃, CF₂CF₃, aryl, phenyl, F, Cl, Br, I, alkenyl or OH;
   A is a ring selected from: 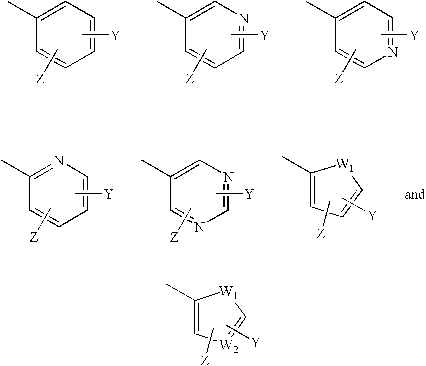
   B is a ring selected from: 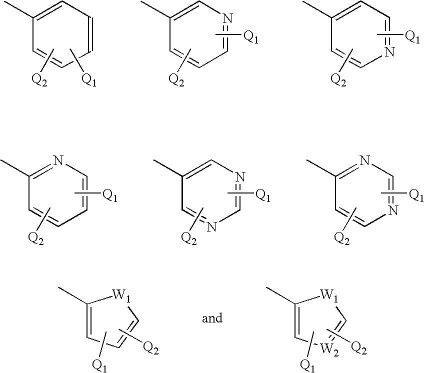
wherein
   A and B cannot simultaneously be a benzene ring;
   Z is NO₂, CN, COOH, COR, NHCOR or CONHR;
   Y is CF₃, F, I, Br, Cl, CN CR₃ or SnR₃;
   Q₁ and Q₂ are independently of each other a hydrogen, alkyl, F, Cl, Br, I, CF₃, CN CR₃, SnR₃, NR₂, NHCOCH₃, NHCOCF₃, NHCOR, NHCONHR, NHCOOR, OCONHR, CONHR, NHCSCH₃, NHCSCF₃, NHCSR NHSO₂CH₃, NHSO₂R, OR, COR, OCOR, OSO₂R, SO₂R, SR, NCS, SCN, NCO, OCN, 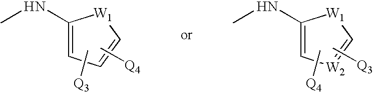
   Q₃ and Q₄ are independently of each other a hydrogen, alkyl, F, Cl, Br, I, CF₃, CN CR₃, SnR₃, NR₂, NHCOCH₃, NHCOCF₃, NHCOR, NHCONHR, NHCOOR, OCONHR, CONHR, NHCSCH₃, NHCSCF₃, NHCSR NHSO₂CH₃, NHSO₂R, OR, COR, OCOR, OSO₂R, SO₂R, SR, NCS, SCN, NCO or OCN;
   W₁ is O, NH, NR, NO or S; and
   W₂ is N or NO;
or its analog, derivative, isomer, metabolite, pharmaceutically acceptable salt, pharmaceutical product, hydrate or N-oxide, prodrug, polymorph or crystal of the SARM compound, or any combination thereof.

In another embodiment, the SARM compound that is effective at treating, preventing, suppressing, inhibiting or reducing the incidence of the ADAM-associated condition is a compound represented by the structure of formula IV: 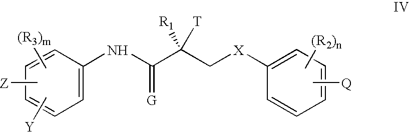
wherein
   X is a bond, O, CH₂, NH, Se, PR, NO or NR;
   G is O or S;
   T is OH, OR, —NHCOCH₃, or NHCOR;
   R is alkyl, haloalkyl, dihaloalkyl, trihaloalkyl, CH₂F, CHF₂, CF₃, CF₂CF₃, aryl, phenyl, F, Cl, Br, I, alkenyl or OH;
   R₁ is CH₃, CH₂F, CHF₂, CF₃, CH₂CH₃, or CF₂CF₃;
   R₂ is F, Cl, Br, I, CH₃, CF₃, OH, CN, NO₂, NHCOCH₃, NHCOCF₃, NHCOR, alkyl, arylalkyl, OR, NH₂, NHR, NR₂ or SR;
   R₃ is F, Cl, Br, I, CN, NO₂, COR, COOH, CONHR, CF₃, SnR₃, or R₃ together with the benzene ring to which it is attached forms a fused ring system represented by the structure: 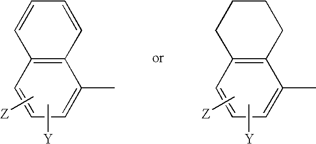
   Z is NO₂, CN, COR, COOH, or CONHR;
   Y is CF₃. F, Br, Cl, I, CN, or SnR₃;
   Q is H, alkyl, F, Cl, Br, I, CF₃, CN CR₃, SnR₃, NR₂, NHCOCH₃, NHCOCF₃, NHCOR, NHCONHR, NHCOOR, OCONHR, CONHR, NHCSCH₃, NHCSCF₃, NHCSR NHSO₂CH₃, NHSO₂R, OH, OR, COR, OCOR, OSO₂R, SO₂R, SR, NCS, SCN, NCO, OCN; or Q together with the benzene ring to which it is attached is a fused ring system represented by structure A, B or C: 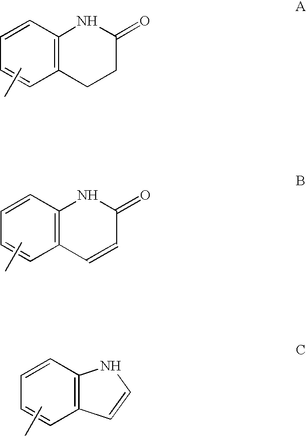
   n is an integer of 1-4; and
   m is an integer of 1-3;
or its analog, derivative, isomer, metabolite, pharmaceutically acceptable salt, pharmaceutical product, hydrate or N-oxide, prodrug, polymorph or crystal of the SARM compound, or any combination thereof.

In another embodiment, the SARM compound that is effective at treating, preventing, suppressing, inhibiting or reducing the incidence of the ADAM-associated condition is a compound represented by the structure of formula V: 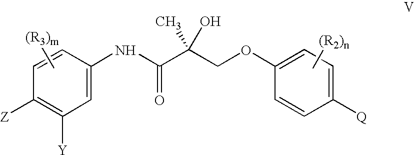
wherein
   R₂ is F, Cl, Br, I, CH₃, CF₃, OH, CN, NO₂, NHCOCH₃, NHCOCF₃, NHCOR, alkyl, arylalkyl, OR, NH₂, NHR, NR₂ or SR;
   R₃ is F, Cl, Br, I, (CN, NO₂, COR, COOH, CONHR, CF₃, SnR₃, or R₃ together with the benzene ring to which it is attached forms a fused ring system represented by the structure: 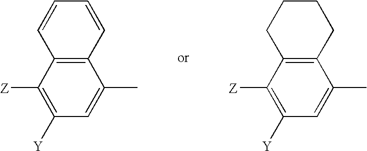
   R is alkyl, haloalkyl, dihaloalkyl, trihaloalkyl, CH₂F, CHF₂, CF₃, CF₂CF₃, aryl, phenyl, F, Cl, Br, I, alkenyl or OH;
   Z is NO₂, CN, COR, COOH, or CONHR;
   Y is CF₃, F, Br, Cl, I, CN, or SnR₃;
   Q is H, alkyl, F, Cl, Br, I, CF₃, CN CR₃, SnR₃, NR₂, NHCOCH₃, NHCOCF₃, NHCOR, NHCONHR, NHCOOR, OCONHR, CONHR, NHCSCH₃, NHCSCF₃, NHCSR NHSO₂CH₃, NHSO₂R, OH, OR, COR, OCOR, OSO₂R, SO₂R, SR, NCS, SCN, NCO, OCN; or Q together with the benzene ring to which it is attached is a fused ring system represented by structure A, B or C: 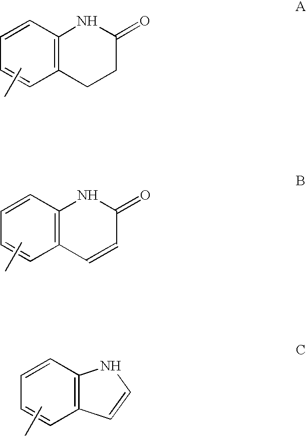
   n is an integer of 1-4; and
   m is an integer of 1-3;
or its analog, derivative, isomer, metabolite, pharmaceutically acceptable salt, pharmaceutical product, hydrate or N-oxide, prodrug, polymorph or crystal of the SARM compound, or any combination thereof.

In another embodiment, the SARM compound that is effective at treating, preventing, suppressing, inhibiting or reducing the incidence of the ADAM-associated condition is a compound represented by the structure of formula VI, or its analog, derivative, isomer, metabolite, pharmaceutically acceptable salt, pharmaceutical product, hydrate or N-oxide, prodrug, polymorph or crystal of the SARM compound, or any combination thereof

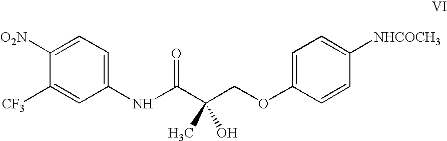

In another embodiment, the SARM compound that is effective at treating, preventing, suppressing, inhibiting or reducing the incidence of the ADAM-associated condition is a compound represented by the structure of formula VII, or its analog, derivative, isomer, metabolite, pharmaceutically acceptable salt, pharmaceutical product, hydrate or N-oxide, prodrug, polymorph or crystal of the SARM compound, or any combination thereof

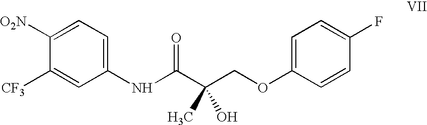

In one embodiment, the SARM is an androgen receptor agonist. In another embodiment, the SARM is an androgen receptor antagonist. In another embodiment, the SARM has an agonistic effect muscle or bone. In another embodiment, the SARM has no effect on muscle or bone. In another embodiment, the SARM has no effect or an antagonistic effect on prostate. In another embodiment, the SARM has an agonistic effect muscle or bone and no effect or an antagonistic effect on prostate. In another embodiment, the SARM has no effect on muscle or bone and has no effect or an antagonistic effect on prostate. In another embodiment, the SARM penetrates the central nervous system (CNS). In another embodiment, the SARM does not penetrate the central nervous system (CNS).

In one embodiment, the ADAM-associated condition is sexual dysfunction. In another embodiment, the ADAM-associated condition is decreased sexual libido. In another embodiment, the ADAM-associated condition is erectile dysfunction. In another embodiment, the ADAM-associated condition is hypogonadism. In another embodiment, the ADAM-associated condition is sarcopenia. In another embodiment, the ADAM-associated condition is osteopenia. In another embodiment, the ADAM-associated condition is osteoporosis. In another embodiment, the ADAM-associated condition is benign prostate hyperplasia. In another embodiment, the ADAM-associated condition is prostate cancer. In another embodiment, the ADAM-associated condition comprises alterations in cognition and mood. In another embodiment, the ADAM-associated condition is depression. In another embodiment, the ADAM-associated condition is anemia. In another embodiment, the ADAM-associated condition is hair loss. In another embodiment, the ADAM-associated condition is obesity. In another embodiment, the ADAM-associated condition is any combination of the conditions recited hereinabove.

The present invention provides a safe and effective method of treating, preventing, suppressing, inhibiting or reducing the incidence of ADAM-associated conditions and is particularly useful in treating male subjects suffering from symptoms and signs of sexual dysfunction, decreased sexual libido, erectile dysfunction, hypogonadism, sarcopenia, osteopenia, osteoporosis, alterations in cognition and mood, depression, anemia, hair loss, obesity, benign prostate hyperplasia and/or prostate cancer

### BRIEF DESCRIPTION OF THE DRAWINGS

FIG. 1: Flowchart of ADAM-associated conditions.

FIG. 2: Schematic illustration of ADAM-associated conditions.

FIG. 3: Androgenic and Anabolic activity of Compound VI and Compound VII in rats. Male rats with normal testicular function (no surgical manipulation) were left untreated (Intact), treated with compound VI (0.5 mg/day), compound VII (0.5 mg/day) or testosterone proprionate (TP, 0.5 mg/day), and the weight of androgen-responsive tissues (prostate—FIG. 3A, semimal vesicles—FIG. 3B, and levator ani muscle—FIG. 3C) was determined.

FIG. 4: Androgenic and Anabolic activity of Compound VI and Compound VII in rats. Male rats received unilateral orchidectomy (Hemi-orchidectomized) and were left untreated (Intact), treated with vehicle alone (PEG 300), Compound VI (0.5 mg/day), Compound VII (0.5 mg/day), or testosterone proprionate (TP, 0.5 mg/day), and the weight of androgen-responsive tissues (prostate—FIG. 4A, semimal vesicles—FIG. 4B, and levator ani muscle—FIG. 4C) was determined.

FIG. 5: Androgenic and Anabolic activity of Compound VI and Compound VII in rats. Male rats received bilateral orchidectomy (Castrated) and were left untreated (Intact), treated with vehicle alone (PEG 300), Compound VI (0.5 mg/day), Compound VII (0.5 mg/day), or testosterone proprionate (TP, 0.5 mg/day), and the weight of androgen-responsive tissues (prostate—FIG. 5A, semimal vesicles—FIG. 5B, and levator ani muscle—FIG. 5C) was determined.

FIG. 6: Dose response Curves. Rats were left untreated, or treated with 0.1, 0.3, 0.5, 0.75 and 1.0 mg/day Compound VI, Compound VII or testosterone propionate (TP), and the weight of androgen-responsive tissues (prostate—FIG. 6A, semimal vesicles—FIG. 6B and levator ani muscle—FIG. 6C) was determined. The results are plotted as percentage of the intact control.

FIG. 7: Effect of testosterone proprionate and Compound VI on mysoin heavy chain (MHC) IIb mRNA expression. FIG. 7A: histogram showing effect of Compound VI on MHC IIb mRNA expression; and FIG. 7B: RT-PCR showing m-RNA expression of MHC IIb.

FIG. 8: Effect of SARMS on Bone Mineral Content (BMC) and Bone Mineral Density (BMD) in female rates after ovariectomy.

FIG. 9: Compound VI increased whole body BMC in a dose-dependent and time-dependent manner.

FIG. 10: Compound VI exerted a protective effect at both the L2-L4 vertebra and proximal femur.

FIG. 11: Compound VI increased biomechanical strength of the L5 vertebra and femur.

FIG. 12: Compound VI increased cortical thickness in the femoral mid-shaft.

### DETAILED DESCRIPTION OF THE INVENTION

The present invention provides a method of treating, preventing, suppressing, inhibiting or reducing the incidence of an Androgen Decline in Aging Male (ADAM)-associated condition in a male subject, by administering to the subject a selective androgen receptor modulator (SARM) compound and/or its analog, derivative, isomer, metabolite, pharmaceutically acceptable salt, pharmaceutical product, hydrate, N-oxide, prodrug, polymorph, crystal, or any combination thereof. The present invention further provides a method of treating, preventing, suppressing, inhibiting or reducing the incidence of sexual dysfunction, decreased sexual libido, erectile dysfunction, hypogonadism, sarcopenia, osteopenia, osteoporosis, alterations in cognition and mood, depression, anemia, hair loss, obesity, benign prostate hyperplasia and/or prostate cancer due to ADAM in a male subject, by administering to the subject a selective androgen receptor modulator (SARM) compound and/or its analog, derivative, isomer, metabolite, pharmaceutically acceptable salt, pharmaceutical product, hydrate, N-oxide, prodrug, polymorph, crystal, or any combination thereof. In one embodiment, the male subject is an aging male subject.

Thus, in one embodiment, this invention relates to a method of treating a male subject suffering from an Androgen Decline in Aging Male (ADAM)-associated condition, comprising the step of administering to the subject a selective androgen receptor modulator (SARM) compound. In another embodiment, the method comprises administering an analog, derivative, isomer, metabolite, pharmaceutically acceptable salt, pharmaceutical product, hydrate or N-oxide, prodrug, polymorph or crystal of the SARM compound, or any combination thereof. In one embodiment, the male subject is an aging male subject.

In another embodiment, the present invention provides a method of preventing, suppressing, inhibiting or reducing the incidence of an ADAM-associated condition in a male subject, comprising the step of administering to the subject a selective androgen receptor modulator (SARM) compound. In another embodiment, the method comprises administering an analog, derivative, isomer, metabolite, pharmaceutically acceptable salt, pharmaceutical product, hydrate or N-oxide, prodrug, polymorph or crystal of the SARM compound, or any combination thereof. In one embodiment, the male subject is an aging male subject.

In another embodiment, the present invention provides a method of treating a male subject suffering from sexual dysfunction, decreased sexual libido, erectile dysfunction, hypogonadism, sarcopenia, osteopenia, osteoporosis, alterations in cognition and mood, depression, anemia, hair loss, obesity, benign prostate hyperplasia and/or prostate cancer due to Androgen Decline in an Aging Male (ADAM), comprising the step of administering to the subject a selective androgen receptor modulator (SARM) compound. In another embodiment, the method comprises administering an analog, derivative, isomer, metabolite, pharmaceutically acceptable salt, pharmaceutical product, hydrate or N-oxide, prodrug, polymorph or crystal of the SARM compound, or any combination thereof. In one embodiment, the male subject is an aging male subject.

In another embodiment, the present invention provides a method of preventing, suppressing, inhibiting or reducing the incidence of an ADAM-associated condition selected from sexual dysfunction, decreased sexual libido, erectile dysfunction, hypogonadism, sarcopenia, osteopenia, osteoporosis, alterations in cognition and mood, depression, anemia, hair loss, obesity, benign prostate hyperplasia and/or prostate cancer in a male subject, comprising the step of administering to the subject a selective androgen receptor modulator (SARM) compound. In another embodiment, the method comprises administering an analog, derivative, isomer, metabolite, pharmaceutically acceptable salt, pharmaceutical product, hydrate or N-oxide, prodrug, polymorph or crystal of the SARM compound, or any combination thereof. In one embodiment, the male subject is an aging male subject.

In one embodiment, the SARM compound that is effective at treating, preventing, suppressing, inhibiting or reducing the incidence of the ADAM-associated condition is a compound represented by the structure of formula I: 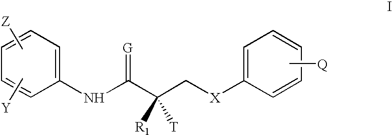
wherein
   G is O or S;
   X is a bond, O, CH₂, NH, Se, PR, NO or NR;
   T is OH, OR, —NHCOCH₃, or NHCOR
   Z is NO₂, CN, COOH, COR, NHCOR or CONHR;
   Y is CF₃, F, I, Br, Cl, CN, CR₃ or SnR₃;
   Q is alkyl, F, Cl, Br, I, CF₃, CN CR₃, SnR₃, NR₂, NHCOCH₃, NHCOCF₃, NHCOR, NHCONHR, NHCOOR, OCONHR, CONHR, NHCSCH₃, NHCSCF₃, NHCSR NHSO₂CH₃, NHSO₂R, OR, COR, OCOR, OSO₂R, SO₂R, SR, NCS, SCN, NCO, OCN; or Q together with the benzene ring to which it is attached is a fused ring system represented by structure A, B or C: 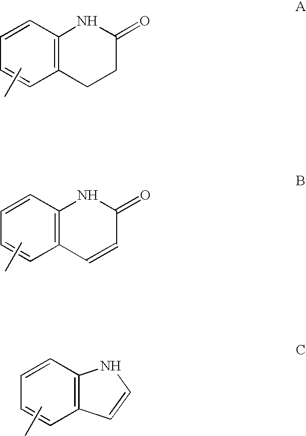
   R is alkyl, haloalkyl, dihaloalkyl, trihaloalkyl, CH₂F, CHF₂, CF₃, CF₂CF₃, aryl, phenyl, F, Cl, Br, I, alkenyl or OH; and
   R₁ is CH₃, CH₂F, CHF₂, CF₃, CH₂CH₃, or CF₂CF₃.

In one embodiment, the SARM is an analog of the compound of formula I. In another embodiment, the SARM is a derivative of the compound of formula I. In another embodiment, the SARM is an isomer of the compound of formula I. In another embodiment, the SARM is a metabolite of the compound of formula I. In another embodiment, the SARM is a pharmaceutically acceptable salt of the compound of formula I. In another embodiment, the SARM is a pharmaceutical product of the compound of formula I. In another embodiment, the SARM is a hydrate of the compound of formula I. In another embodiment, the SARM is an N-oxide of the compound of formula I. In another embodiment, the SARM is a crystal of the compound of formula I. In another embodiment, the SARM is a polymorph of the compound of formula I. In another embodiment, the SARM is a prodrug of the compound of formula I. In another embodiment, the SARM is a combination of any of an analog, derivative, metabolite, isomer, pharmaceutically acceptable salt, pharmaceutical product, hydrate, N-oxide, crystal, polymorph or prodrug of the compound of formula I.

In one embodiment, the SARM compound is a compound of formula I wherein X is O. In one embodiment, the SARM compound is a compound of formula I wherein G is O. In another embodiment, the SARM compound is a compound of formula I wherein Z is NO₂. In another embodiment, the SARM compound is a compound of formula I wherein Z is CN. In another embodiment, the SARM compound is a compound of formula I wherein Y is CF₃. In another embodiment, the SARM compound is a compound of formula I wherein Q is NHCOCH₃. In another embodiment, the SARM compound is a compound of formula I wherein Q is F. In another embodiment, the SARM compound is a compound of formula I wherein T is OH. In another embodiment, the SARM compound is a compound of formula I wherein R₁ is CH₃.

The substituents Z and Y can be in any position of the ring carrying these substituents (hereinafter “A ring”). In one embodiment, the substituent Z is in the para position of the A ring. In another embodiment, the substituent Y is in the meta position of the A ring. In another embodiment, the substituent Z is in the para position of the A ring and substituent Y is in the meta position of the A ring.

The substituent Q can be in any position of the ring carrying this substituent (hereinafter “B ring”). In one embodiment, the substituent Q is in the para position of the B ring. In another embodiment, the substituent Q is NHCOCH₃ and is in the para position of the B ring. In another embodiment, the substituent Q is F and is in the para position of the B ring.

In another embodiment, the SARM compound that is effective at treating, preventing, suppressing, inhibiting or reducing the incidence of the ADAM-associated condition is a compound represented by the structure of formula II: 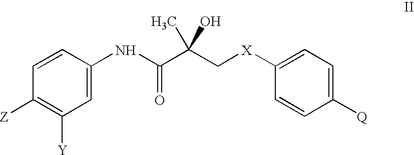
wherein
   X is a bond, O, CH₂, NH, Se, PR, NO or NR;
   Z is NO₂, CN, COOH, COR, NHCOR or CONHR;
   Y is CF₃, F, I, Br, Cl, CN, CR₃ or SnR₃;
   Q is alkyl, F, Cl, Br, I, CF₃, CN CR₃, SnR₃, NR₂, NHCOCH₃, NHCOCF₃, NHCOR, NHCONHR, NHCOOR, OCONHR, CONHR, NHCSCH₃, NHCSCF₃, NHCSR NHSO₂CH₃, NHSO₂R, OR, COR, OCOR, OSO₂R, SO₂R, SR, NCS, SCN, NCO, OCN; or Q together with the benzene ring to which it is attached is a fused ring system represented by structure A, B or C: 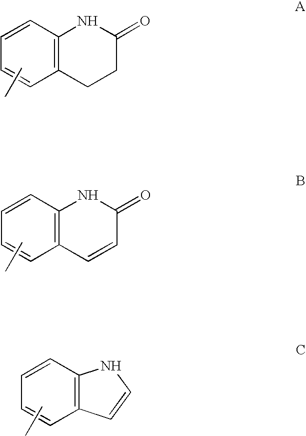
   R is alkyl, haloalkyl, dihaloalkyl, trihaloalkyl, CH₂F, CHF₂, CF₃, CF₂CF₃, aryl, phenyl, F, Cl, Br, I, alkenyl or OH.

In one embodiment, the SARM is an analog of the compound of formula II. In another embodiment, the SARM is a derivative of the compound of formula II. In another embodiment, the SARM is an isomer of the compound of formula II. In another embodiment, the SARM is a metabolite of the compound of formula II. In another embodiment, the SARM is a pharmaceutically acceptable salt of the compound of formula II. In another embodiment, the SARM is a pharmaceutical product of the compound of formula II. In another embodiment, the SARM is a hydrate of the compound of formula II. In another embodiment, the SARM is an N-oxide of the compound of formula II. In another embodiment, the SARM is a crystal of the compound of formula II. In another embodiment, the SARM is a polymorph of the compound of formula II. In another embodiment, the SARM is a prodrug of the compound of formula II. In another embodiment, the SARM is a combination of any of an analog, derivative, metabolite, isomer, pharmaceutically acceptable salt, pharmaceutical product, hydrate, N-oxide, crystal, polymorph or prodrug of the compound of formula II.

In one embodiment, the SARM compound is a compound of formula II wherein X is O. In another embodiment, the SARM compound is a compound of formula II wherein Z is NO₂. In another embodiment, the SARM compound is a compound of formula II wherein Z is CN. In another embodiment, the SARM compound is a compound of formula II wherein Y is CF₃. In another embodiment, the SARM compound is a compound of formula II wherein Q is NHCOCH₃. In another embodiment, the SARM compound is a compound of formula II wherein Q is F.

In another embodiment, the SARM compound that is effective at treating, preventing, suppressing, inhibiting or reducing the incidence of the ADAM-associated condition is a compound represented by the structure of formula III: 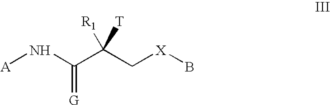
wherein
   X is a bond, O, CH₂, NH, Se, PR, NO or NR;
   G is O or S;
   R₁ is CH₃, CH₂F, CHF₂, CF₃, CH₂CH₃, or CF₂CF₃;
   T is OH, OR, —NHCOCH₃, or NHCOR;
   R is alkyl, haloalkyl, dihaloalkyl, trihaloalkyl, CH₂F, CHF₂, CF₃, CF₂CF₃, aryl, phenyl, F, Cl, Br, I, alkenyl or OH;
   A is a ring selected from: 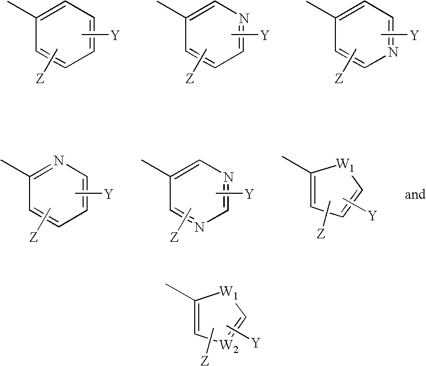
   B is a ring selected from: 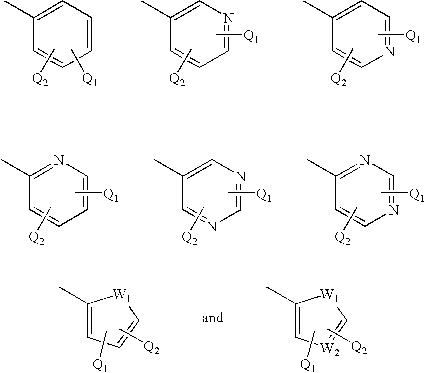
wherein
   A and B cannot simultaneously be a benzene ring;
   Z is NO₂, CN, COOH, COR, NHCOR or CONHR;
   Y is CF₃, F, I, Br, Cl, CN CR₃ or SnR₃;
   Q₁ and Q₂ are independently of each other a hydrogen, alkyl, F, Cl, Br, I, CF₃, CN CR₃, SnR₃, NR₂, NHCOCH₃, NHCOCF₃, NHCOR, NHCONHR, NHCOOR, OCONHR, CONHR, NHCSCH₃, NHCSCF₃, NHCSR NHSO₂CH₃, NHSO₂R, OR, COR, OCOR, OSO₂R, SO₂R, SR, NCS, SCN, NCO, OCN, 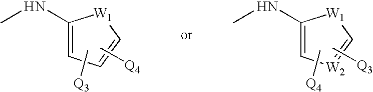
   Q₃ and Q₄ are independently of each other a hydrogen, alkyl, F, Cl, Br, I, CF₃, CN CR₃, SnR₃, NR₂, NHCOCH₃, NHCOCF₃, NHCOR, NHCONHR, NHCOOR, OCONHR, CONHR, NHCSCH₃, NHCSCF₃, NHCSR NHSO₂CH₃, NHSO₂R, OR, COR, OCOR, OSO₂R, SO₂R, SR, NCS, SCN, NCO or OCN;
   W₁ is O, NH, NR, NO or S; and
   W₂ is N or NO.

In one embodiment, the SARM is an analog of the compound of formula III. In another embodiment, the SARM is a derivative of the compound of formula III. In another embodiment, the SARM is an isomer of the compound of formula III. In another embodiment, the SARM is a metabolite of the compound of formula III. In another embodiment, the SARM is a pharmaceutically acceptable salt of the compound of formula III. In another embodiment, the SARM is a pharmaceutical product of the compound of formula III. In another embodiment, the SARM is a hydrate of the compound of formula III. In another embodiment, the SARM is an N-oxide of the compound of formula III. In another embodiment, the SARM is a crystal of the compound of formula III. In another embodiment, the SARM is a polymorph of the compound of formula III. In another embodiment, the SARM is a prodrug of the compound of formula III. In another embodiment, the SARM is a combination of any of an analog, derivative, metabolite, isomer, pharmaceutically acceptable salt, pharmaceutical product, hydrate, N-oxide, crystal, polymorph or prodrug of the compound of formula III.

In one embodiment, the SARM compound is a compound of formula III wherein X is O. In another embodiment, the SARM compound is a compound of formula III wherein G is O. In another embodiment, the SARM compound is a compound of formula I wherein T is OH. In another embodiment, the SARM compound is a compound of formula III wherein R₁ is CH₃. In another embodiment, the SARM compound is a compound of formula III wherein Z is NO₂. In another embodiment, the SARM compound is a compound of formula III wherein Z is CN. In another embodiment, the SARM compound is a compound of formula III wherein Y is CF₃. In another embodiment, the SARM compound is a compound of formula III wherein Q₁ is NHCOCH₃ In another embodiment, the SARM compound is a compound of formula III wherein Q₁ is F.

The substituents Z and Y can be in any position of the ring carrying these substituents (hereinafter “A ring”). In one embodiment, the substituent Z is in the para position of the A ring. In another embodiment, the substituent Y is in the meta position of the A ring. In another embodiment, the substituent Z is in the para position of the A ring and substituent Y is in the meta position of the A ring.

The substituents Q₁ and Q₂ can be in any position of the ring carrying these substituents (hereinafter “B ring”). In one embodiment, the substitutent Q₁ is in the para position of the B ring. In another embodiment, the subsituent is Q₂ is H. In another embodiment, the substitutent Q₁ is in the para position of the B ring and the subsituent is Q₂ is H. In another embodiment, the substitutent Q₁ is NHCOCH₃ and is in the para position of the B ring, and the substituent is Q₂ is H.

In another embodiment, the SARM compound that is effective at treating, preventing, suppressing, inhibiting or reducing the incidence of the ADAM-associated condition is a compound represented by the structure of formula IV: 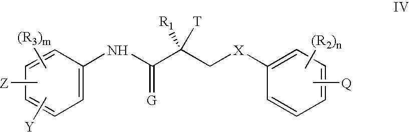
wherein
   X is a bond, O, CH₂, NH, Se, PR, NO or NR;
   G is O or S;
   T is OH, OR, —NHCOCH₃, or NHCOR;
   R is alkyl, haloalkyl, dihaloalkyl, trihaloalkyl, CH₂F, CHF₂, CF₃, CF₂CF₃, aryl, phenyl, F, Cl, Br, I, alkenyl or OH;
   R₁ is CH₃, CH₂F, CHF₂, CF₃, CH₂CH₃, or CF₂CF₃;
   R₂ is F, Cl, Br, I, CH₃, CF₃, OH, CN, NO₂, NHCOCH₃, NHCOCF₃, NHCOR, alkyl, arylalkyl, OR, NH₂, NHR, NR₂ or SR;
   R₃ is F, Cl, Br, I, CN, NO₂, COR, COOH, CONHR, CF₃, SnR₃, or R₃ together with the benzene ring to which it is attached forms a fused ring system represented by the structure: 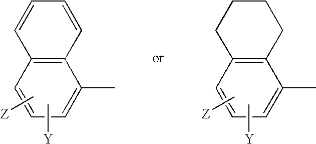
   Z is NO₂, CN, COR, COOH, or CONHR;
   Y is CF₃, F, Br, Cl, I, CN, or SnR₃;
   Q is H, alkyl, F, Cl, Br, I, CF₃, CN CR₃, SnR₃, NR₂, NHCOCH₃, NHCOCF₃, NHCOR, NHCONHR, NHCOOR, OCONHR, CONHR, NHCSCH₃, NHCSCF₃, NHCSR NHSO₂CH₃, NHSO₂R, OH, OR, COR, OCOR, OSO₂R, SO₂R, SR, NCS, SCN, NCO, OCN; or Q together with the benzene ring to which it is attached is a fused ring system represented by structure A, B or C: 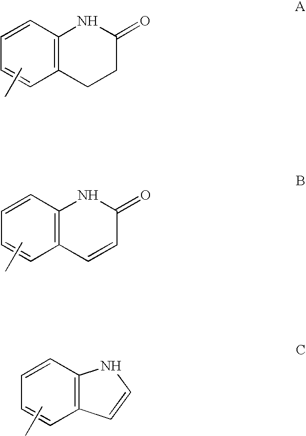
   n is an integer of 1-4; and
   m is an integer of 1-3.

In one embodiment, the SARM is an analog of the compound of formula IV. In another embodiment, the SARM is a derivative of the compound of formula IV. In another embodiment, the SARM is an isomer of the compound of formula IV. In another embodiment, the SARM is a metabolite of the compound of formula IV. In another embodiment, the SARM is a pharmaceutically acceptable salt of the compound of formula IV. In another embodiment, the SARM is a pharmaceutical product of the compound of formula IV. In another embodiment, the SARM is a hydrate of the compound of formula IV. In another embodiment, the SARM is an N-oxide of the compound of formula IV. In another embodiment, the SARM is a crystal of the compound of formula IV. In another embodiment, the SARM is a polymorph of the compound of formula IV. In another embodiment, the SARM is a prodrug of the compound of formula IV. In another embodiment, the SARM is a combination of any of an analog, derivative, metabolite, isomer, pharmaceutically acceptable salt, pharmaceutical product, hydrate, N-oxide, crystal, polymorph or prodrug of the compound of formula IV.

In one embodiment, the SARM compound is a compound of formula IV wherein X is O. In another embodiment, the S ARM compound is a compound of formula IV wherein G is O. In another embodiment, the SARM compound is a compound of formula IV wherein Z is NO₂. In another embodiment, the SARM compound is a compound of formula IV wherein Z is CN. In another embodiment, the SARM compound is a compound of formula IV wherein Y is CF₃. In another embodiment, the SARM compound is a compound of formula IV wherein Q is NHCOCH₃. In another embodiment, the SARM compound is a compound of formula IV wherein Q is F. In another embodiment, the SARM compound is a compound of formula IV wherein T is OH. In another embodiment, the SARM compound is a compound of formula IV wherein R₁ is CH₃. In another embodiment, the SARM compound is a compound of formula IV wherein Q is F and R₂ is CH₃. In another embodiment, the SARM compound is a compound of formula IV wherein Q is F and R₂ is Cl.

The substituents Z, Y and R₃ can be in any position of the ring carrying these substituents (hereinafter “A ring”). In one embodiment, the substituent Z is in the para position of the A ring. In another embodiment, the substituent Y is in the meta position of the A ring. In another embodiment, the substituent Z is in the para position of the A ring and substituent Y is in the meta position of the A ring.

The substituents Q and R₂ can be in any position of the ring carrying these substituents (hereinafter “B ring”). In one embodiment, the substitutent Q is in the para position of the B ring. In another embodiment, the substitutent Q is in the para position of the B ring. In another embodiment, the substitutent Q is NHCOCH₃ and is in the para position of the B ring.

As contemplated herein, when the integers m and n are greater than one, the substituents R₂ and R₃ are not limited to one particular substituent, and can be any combination of the substituents listed above.

In another embodiment, the SARM compound that is effective at treating, preventing, suppressing, inhibiting or reducing the incidence of the ADAM-associated condition is a compound represented by the structure of formula V: 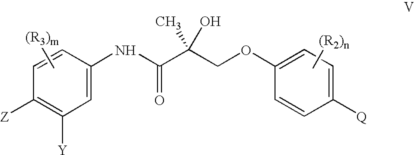
wherein
   R₂ is F, Cl, Br, I, CH₃, CF₃, OH, CN, NO₂, NHCOCH₃, NHCOCF₃, NHCOR, alkyl, arylalkyl, OR, NH₂, NHR, NR₂ or SR;
   R₃ is F, Cl, Br, I, CN, NO₂, COR, COOH, CONHR, CF₃, SnR₃, or R₃ together with the benzene ring to which it is attached forms a fused ring system represented by the structure: 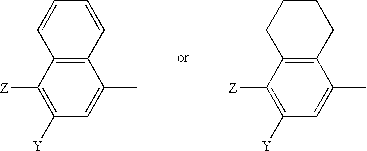
   R is alkyl, haloalkyl, dihaloalkyl, trihaloalkyl, CH₂F, CHF₂, CF₃, CF₂CF₃, aryl, phenyl, F, Cl, Br, I, alkenyl or OH;
   Z is NO₂, CN, COR, COOH, or CONHR;
   Y is CF₃, F, Br, Cl, I, CN, or SnR₃;
   Q is H, alkyl, F, Cl, Br, I, CF₃, CN CR₃, SnR₃, NR₂, NHCOCH₃, NHCOCF₃, NHCOR, NHCONHR, NHCOOR, OCONHR, CONHR, NHCSCH₃, NHCSCF₃, NHCSR NHSO₂CH₃, NHSO₂R, OH, OR, COR, OCOR, OSO₂R, SO₂R, SR, NCS, SCN, NCO, OCN; or Q together with the benzene ring to which it is attached is a fused ring system represented by structure A, B or C: 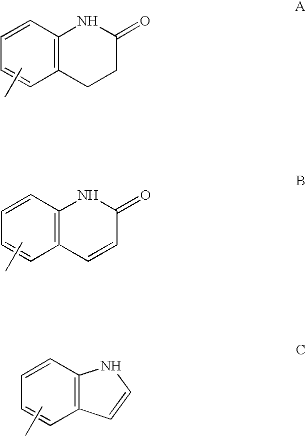
   n is an integer of 1-4; and
   m is an integer of 1-3.

In one embodiment, the SARM is an analog of the compound of formula V. In another embodiment, the SARM is a derivative of the compound of formula V. In another embodiment, the SARM is an isomer of the compound of formula V. In another embodiment, the SARM is a metabolite of the compound of formula V. In another embodiment, the SARM is a pharmaceutically acceptable salt of the compound of formula V. In another embodiment, the SARM is a pharmaceutical product of the compound of formula V. In another embodiment, the SARM is a hydrate of the compound of formula V. In another embodiment, the SARM is an N-oxide of the compound of formula V. In another embodiment, the SARM is a crystal of the compound of formula V. In another embodiment, the SARM is a polymorph of the compound of formula V. In another embodiment, the SARM is a prodrug of the compound of formula V. In another embodiment, the SARM is a combination of any of an analog, derivative, metabolite, isomer, pharmaceutically acceptable salt, pharmaceutical product, hydrate, N-oxide, crystal, polymorph or prodrug of the compound of formula V.

In another embodiment, the SARM is a compound of formula V wherein Z is NO₂. In another embodiment, the SARM is a compound of formula V wherein Z is CN. In another embodiment, the SARM is a compound of formula V wherein Y is CF₃. In another embodiment, the SARM is a compound of formula V wherein Q is NHCOCH₃. In another embodiment, the SARM is a compound of formula V wherein Q is F. In another embodiment, the SARM is a compound of formula V wherein Q is F and R₂ is CH₃ In another embodiment, the SARM is a compound of formula V wherein Q is F and R₂ is Cl.

The substituents Z, Y and R₃ can be in any position of the A ring, and the substituents Q and R₂ can be in any position of B ring, as discussed above for compound IV. Furthermore, as discussed above, when the integers m and n are greater than one, the substituents R₂ and R₃ are not limited to one particular substituent, and can be any combination of the substituents listed above.

In another embodiment, the SARM compound that is effective at treating, preventing, suppressing, inhibiting or reducing the incidence of the ADAM-associated condition is a compound represented by the structure of formula VI.

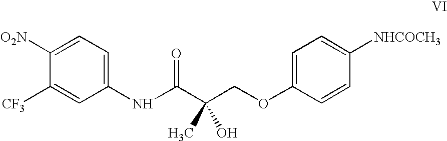

In one embodiment, the SARM is an analog of the compound of formula VI. In another embodiment, the SARM is a derivative of the compound of formula VI. In another embodiment, the SARM is an isomer of the compound of formula VI. In another embodiment, the SARM is a metabolite of the compound of formula VI. In another embodiment, the SARM is a pharmaceutically acceptable salt of the compound of formula VI. In another embodiment, the SARM is a pharmaceutical product of the compound of formula VI. In another embodiment, the SARM is a hydrate of the compound of formula VI. In another embodiment, the SARM is an N-oxide of the compound of formula VI. In another embodiment, the SARM is a crystal of the compound of formula VI. In another embodiment, the SARM is a polymorph of the compound of formula VI. In another embodiment, the SARM is a prodrug of the compound of formula VI. In another embodiment, the SARM is a combination of any of an analog, derivative, metabolite, isomer, pharmaceutically acceptable salt, pharmaceutical product, hydrate, N-oxide, crystal, polymorph or prodrug of the compound of formula VI.

In another embodiment, the SARM compound that is effective at treating, preventing, suppressing, inhibiting or reducing the incidence of the ADAM-associated condition is a compound represented by the structure of formula VII.

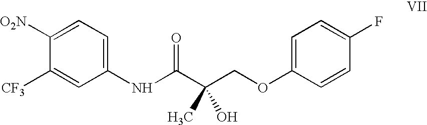

In one embodiment, the SARM is an analog of the compound of formula VII. In another embodiment, the SARM is a derivative of the compound of formula VII. In another embodiment, the SARM is an isomer of the compound of formula VII. In another embodiment, the SARM is a metabolite of the compound of formula VII. In another embodiment, the SARM is a pharmaceutically acceptable salt of the compound of formula VII. In another embodiment, the SARM is a pharmaceutical product of the compound of formula VII. In another embodiment, the SARM is a hydrate of the compound of formula VII. In another embodiment, the SARM is an N-oxide of the compound of formula VII. In another embodiment, the SARM is a crystal of the compound of formula VII. In another embodiment, the SARM is a polymorph of the compound of formula VII. In another embodiment, the SARM is a prodrug of the compound of formula VII. In another embodiment, the SARM is a combination of any of an analog, derivative, metabolite, isomer, pharmaceutically acceptable salt, pharmaceutical product, hydrate, N-oxide, crystal, polymorph or prodrug of the compound of formula VII.

The substituent R is defined herein as an alkyl, haloalkyl, dihaloalkyl, trihaloalkyl, CH₂F, CHF₂, CF₃, CF₂CF₃; aryl, phenyl, F, Cl, Br, I, alkenyl, or hydroxyl (OH).

An “alkyl” group refers to a saturated aliphatic hydrocarbon, including straight-chain, branched-chain and cyclic alkyl groups. In one embodiment, the alkyl group has 1-12 carbons. In another embodiment, the alkyl group has 1-7 carbons. In another embodiment, the alkyl group has 1-6 carbons. In another embodiment, the alkyl group has 1-4 carbons. The alkyl group may be unsubstituted or substituted by one or more groups selected from halogen (e.g. F, Cl, Br, I), hydroxy, alkoxy carbonyl, amido, alkylamido, dialkylamido, nitro, amino, alkylamino, dialkylamino, carboxyl, thio and thioalkyl.

A “haloalkyl” group refers to an alkyl group as defined above, which is substituted by one or more halogen atoms, e.g. by F, Cl, Br or I. A “halogen” refers to elements of Group VII or the periodic table, e.g. F, Cl, Br or I.

An “aryl” group refers to an aromatic group having at least one carbocyclic aromatic group or heterocyclic aromatic group, which may be unsubstituted or substituted by one or more groups selected from halogen (e.g. F, Cl, Br, I), haloalkyl, hydroxy, alkoxy carbonyl, amido, alkylamido, dialkylamido, nitro, amino, alkylamino, dialkylamino, carboxy or thio or thioalkyl. Nonlimiting examples of aryl rings are phenyl, naphthyl, pyranyl, pyrrolyl, pyrazinyl, pyrimidinyl, pyrazolyl, pyridinyl, furanyl, thiophenyl, thiazolyl, imidazolyl, isoxazolyl, and the like.

A “hydroxyl” group refers to an OH group. An “alkenyl” group refers to a group having at least one carbon to carbon double bond.

An “arylalkyl” group refers to an alkyl bound to an aryl, wherein alkyl and aryl are as defined above. An example of an aralkyl group is a benzyl group.

As contemplated herein, the present invention relates to the use of a SARM compound and/or its analog, derivative, isomer, metabolite, pharmaceutically acceptable salt, pharmaceutical product, hydrate, N-oxide, prodrug, polymorph or crystal or combinations thereof. In one embodiment, the invention relates to the use of an analog of the SARM compound. In another embodiment, the invention relates to the use of a derivative of the SARM compound. In another embodiment, the invention relates to the use of an isomer of the SARM compound. In another embodiment, the invention relates to the use of a metabolite of the SARM compound. In another embodiment, the invention relates to the use of a pharmaceutically acceptable salt of the SARM compound. In another embodiment, the invention relates to the use of a pharmaceutical product of the SARM compound. In another embodiment, the invention relates to the use of a hydrate of the SARM compound. In another embodiment, the invention relates to the use of an N-oxide of the SARM compound. In another embodiment, the invention relates to the use of a prodrug of the SARM compound. In another embodiment, the invention relates to the use of a polymorph of the SARM compound. In another embodiment, the invention relates to the use of a crystal of the SARM compound. In another embodiment, the invention relates to the use of any of a combination of an analog, derivative, isomer, metabolite, pharmaceutically acceptable salt, pharmaceutical product, hydrate, or N-oxide, prodrug, polymorph or crystal of the SARM compounds of the present invention.

As defined herein, the term “isomer” includes, but is not limited to, optical isomers and analogs, structural isomers and analogs, conformational isomers and analogs, and the like.

In one embodiment, this invention encompasses the use of various optical isomers of the SARM compounds. It will be appreciated by those skilled in the art that the SARM compounds of the present invention contain at least one chiral center. Accordingly, the SARM compounds used in the methods of the present invention may exist in, and be isolated in, optically-active or racemic forms. Some compounds may also exhibit polymorphism. It is to be understood that the present invention encompasses any racemic, optically-active, polymorphic, or stereroisomeric form, or mixtures thereof, which form possesses properties useful in the treatment of obesity and related disorders as described herein. In one embodiment, the SARM compounds are the pure (R)-isomers. In another embodiment, the SARM compounds are the pure (S)-isomers. In another embodiment, the SARM compounds are a mixture of the (R) and the (S) isomers. In another embodiment, the SARM compounds are a racemic mixture comprising an equal amount of the (R) and the (S) isomers. It is well known in the art how to prepare optically-active forms (for example, by resolution of the racemic form by recrystallization techniques, by synthesis from optically-active starting materials, by chiral synthesis, or by chromatographic separation using a chiral stationary phase).

The invention includes pharmaceutically acceptable salts of amino-substituted compounds with organic and inorganic acids, for example, citric acid and hydrochloric acid. The invention also includes N-oxides of the amino substituents of the compounds described herein. Pharmaceutically acceptable salts can also be prepared from the phenolic compounds by treatment with inorganic bases, for example, sodium hydroxide. Also, esters of the phenolic compounds can be made with aliphatic and aromatic carboxylic acids, for example, acetic acid and benzoic acid esters.

This invention further includes derivatives of the SARM compounds. The term “derivatives” includes but is not limited to ether derivatives, acid derivatives, amide derivatives, ester derivatives and the like. In addition, this invention further includes hydrates of the SARM compounds. The term “hydrate” includes but is not limited to hemihydrate, monohydrate, dihydrate, trihydrate and the like.

This invention further includes metabolites of the SARM compounds. The term “metabolite” means any substance produced from another substance by metabolism or a metabolic process.

This invention further includes pharmaceutical products of the SARM compounds. The term “pharmaceutical product” means a composition suitable for pharmaceutical use (pharmaceutical composition), as defined herein.

This invention further includes prodrugs of the SARM compounds. The term “prodrug” means a substance which can be converted in-vivo into a biologically active agent by such reactions as hydrolysis, esterification, desterification, activation, salt formation and the like.

This invention further includes crystals of the SARM compounds. Furthermore, this invention provides polymorphs of the SARM compounds. The term “crystal” means a substance in a crystalline state. The term “polymorph” refers to a particular crystalline state of a substance, having particular physical properties such as X-ray diffraction, IR spectra, melting point, and the like.

Biological Activity of Selective Androgen Receptor Modulator Compounds

Selective androgen receptor modulator (SARM) compounds are a novel class of androgen receptor targeting agents (“ARTA”), that have previously been shown to be useful for a) male contraception; b) treatment of a variety of hormone-related conditions, for example conditions associated with Androgen Decline in Aging Male (ADAM), such as fatigue, depression, decreased libido, sexual dysfunction, erectile dysfunction, hypogonadism, osteoporosis, hair loss, anemia, obesity, sarcopenia, osteopenia, osteoporosis, benign prostate hyperplasia, alterations in mood and cognition and prostate cancer; c) treatment of conditions associated with Androgen Decline in Female (ADIF), such as sexual dysfunction, decreased sexual libido, hypogonadism, sarcopenia, osteopenia, osteoporosis, alterations in cognition and mood, depression, anemia, hair loss, obesity, endometriosis, breast cancer, uterine cancer and ovarian cancer; d) treatment and/or prevention of acute and/or chronic muscular wasting conditions; e) preventing and/or treating dry eye conditions; f) oral androgen replacement therapy; g) decreasing the incidence of, halting or causing a regression of prostate cancer; and/or h) inducing apoptosis in a cancer cell.

As contemplated herein, the SARM compounds of the present invention as useful in treating, preventing, suppressing, inhibiting or reducing the incidence of an Androgen Decline in Aging Male (ADAM)-associated condition in a male subject. In one embodiment, the male subject is an aging male subject, as defined herein.

In one embodiment, the ADAM-associated condition is sexual dysfunction. In another embodiment, the ADAM-associated condition is decreased sexual libido. The term “libido, as used herein, means sexual desire.

In another embodiment, the ADAM-associated condition is erectile dysfunction. The term “erectile”, as used herein, means capable of being erected. An erectile tissue is a tissue which is capable of being greatly dilated and made rigid by the distension of the numerous blood vessels which it contains.

In another embodiment, the ADAM-associated condition is hypogonadism. “Hypogonadism” is a condition resulting from or characterised by abnormally decreased functional activity of the gonads, with retardation of growth and sexual development.

In another embodiment, the ADAM-associated condition is sarcopenia. In another embodiment, the ADAM-associated condition is osteopenia. “Osteopenia” refers to decreased calcification or density of bone. This is a term which encompasses all skeletal systems in which such a condition is noted.

In another embodiment, the ADAM-associated condition is osteoporosis. “Osteoporosis” refers to a thinning of the bones with reduction in bone mass due to depletion of calcium and bone protein. Osteoporosis predisposes a person to fractures, which are often slow to heal and heal poorly. Unchecked osteoporosis can lead to changes in posture, physical abnormality, and decreased mobility.

In another embodiment, the ADAM-associated condition is benign prostate hyperplasia. BPH is a nonmalignant enlargement of the prostate gland, and is the most common non-malignant proliferative abnormality found in any internal organ and the major cause of morbidity in the adult male. BPH occurs in over 75% of men over 50 years of age, reaching 88% prevalence by the ninth decade. BPH frequently results in a gradual squeezing of the portion of the urethra which traverses the prostate (prostatic urethra). This causes patients to experience a frequent urge to urinate because of incomplete emptying of the bladder and urgency of urination. The obstruction of urinary flow can also lead to a general lack of control over urination, including difficulty initiating urination when desired, as well as difficulty in preventing urinary flow because of the inability to empty urine from the bladder, a condition known as overflow urinary incontinence, which can lead to urinary obstruction and to urinary failure.

In another embodiment, the ADAM-associated condition is associated with an alternation in cognition and mood. The term “cognition” refers to the process of knowing, specifically the process of being aware, knowing, thinking, learning and judging. Cognition is related to the fields of psychology, linguistics, computer science, neuroscience, mathematics, ethology and philosophy. The term “mood” refers to a temper or state of the mind. As contemplated herein, alterations means any change for the positive or negative, in cognition and/or mood.

In another embodiment, the ADAM-associated condition is depression. The term “depression” refers to an illness that involves the body, mood and thoughts, that affects the way a person eats, sleeps and the way one feels about oneself, and thinks about things. The signs and symptoms of depression include loss of interest in activities, loss of appetite or overeating, loss of emotional expression, an empty mood, feelings of hopelessness, pessimism, guilt or helplessness, social withdrawal, fatigue, sleep disturbances, trouble concentrating, remembering, or making decisions, restlessness, irritability, headaches, digestive disorders or chronic pain.

In another embodiment, the ADAM-associated condition is hair loss. The term “hair loss”, medically known as alopecia, refers to baldness as in the very common type of male-pattern baldness. Baldness typically begins with patch hair loss on the scalp and sometimes progresses to complete baldness and even loss of body hair. Hair loss affects both males and females.

In another embodiment, the ADAM-associated condition is anemia. “Anemia” refers to the condition of having less than the normal number of red blood cells or less than the normal quantity of hemoglobin in the blood. The oxygen-carrying capacity of the blood is, therefore, decreased. Persons with anemia may feel tired and fatigue easily, appear pale, develop palpitations and become usually short of breath. Anemia is caused by four basic factors: a) hemorrhage (bleeding); b) hemolysis (excessive destruction of red blood cells); c) underproduction of red blood cells; and d) not enough normal hemoglobin. There are many forms of anemia, including aplastic anemia, benzene poisoning, Fanconi anemia, hemolytic disease of the newborn, hereditary spherocytosis, iron deficiency anemia, osteopetrosis, pernicious anemia, sickle cell disease, thalassemia, myelodysplastic syndrome, and a variety of bone marrow diseases. As contemplated herein, the SARM compounds of the present invention are useful in preventing and/or treating any one or more of the above-listed forms of anemia.

In another embodiment, the ADAM-associated condition is obesity. “Obesity” refers to the state of being well above one's normal weight. Traditionally, a person is considered to be obese if they are more than 20 percent over their ideal weight. Obesity has been more precisely defined by the National Institute of Health (NIH) as a Body to Mass Index (BMI) of 30 or above. Obesity is often multifactorial, based on both genetic and behavioral factors. Overweight due to obesity is a significant contributor to health problems. It increases the risk of developing a number of diseases including: Type 2 (adult-onset) diabetes; high blood pressure (hypertension); stroke (cerebrovascular accident or CVA); heart attack (myocardial infarction or MI); heart failure (congestive heart failure); cancer (certain forms such as cancer of the prostate and cancer of the colon and rectum); gallstones and gallbladder disease (cholecystitis); Gout and gouty arthritis; osteoarthritis (degenerative arthritis) of the knees, hips, and the lower back; sleep apnea (failure to breath normally during sleep, lowering blood oxygen); and Pickwickian syndrome (obesity, red face, underventilation and drowsiness). As contemplated herein, the term “obesity” includes any one of the above-listed obesity-related conditions and diseases. Thus the SARM compounds of the present invention are useful in preventing and/or treating obesity and any one or more of the above-listed obesity-related conditions and diseases.

In another embodiment, the ADAM-associated condition is prostate cancer. Prostate cancer is one of the most frequently occurring cancers among men in the United States, with hundreds of thousands of new cases diagnosed each year. Over sixty percent of newly diagnosed cases of prostate cancer are found to be pathologically advanced, with no cure and a dismal prognosis. One third of all men over 50 years of age have a latent form of prostate cancer that may be activated into the life-threatening clinical prostate cancer form. The frequency of latent prostatic tumors has been shown to increase substantially with each decade of life from the 50s (5.3-14%) to the 90s (40-80%). The number of people with latent prostate cancer is the same across all cultures, ethnic groups, and races, yet the frequency of clinically aggressive cancer is markedly different. This suggests that environmental factors may play a role in activating latent prostate cancer.

In one embodiment, the male subject which the SARM compounds of the present invention are administered to is an aging male subject. As defined herein, the term “aging” means a process of becoming older. In one embodiment, the aging male is a male over 40 years old. In another embodiment, the aging male is a male over 45 years old. In another embodiment, the aging male is a male over 45 years old. In another embodiment, the aging male is a male over 50 years old. In another embodiment, the aging male is a male over 55 years old. In another embodiment, the aging male is a male over 60 years old. In another embodiment, the aging male is a male over 65 years old. In another embodiment, the aging male is a male over 70 years old. In another embodiment, the aging male is a male over 75 years old.

As contemplated herein, the SARM compounds of the present invention are effective at treating or preventing different ADAM-associated conditions, and may be categorized into subgroups depending on their biological activity. For example, several SARM compounds have an agonistic effect on muscle or bone. Other SARM compounds have no effect on muscle or bone. Other SARM compounds have no effect or an antagonistic effect on prostate. Other SARM compounds are able to penetrate the central nervous system (CNS). Other SARM compounds do not penetrate the central nervous system (CNS).

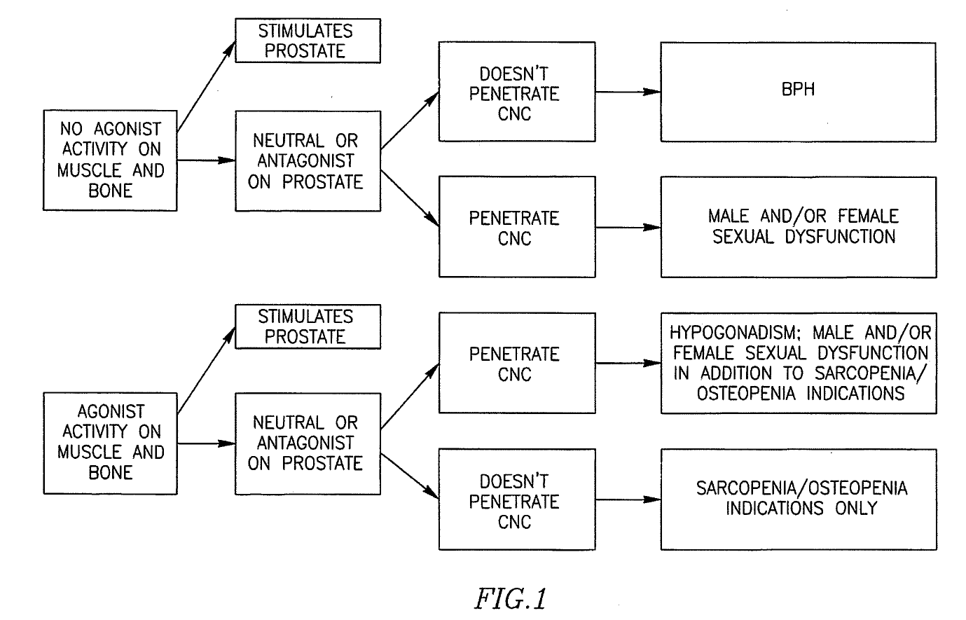

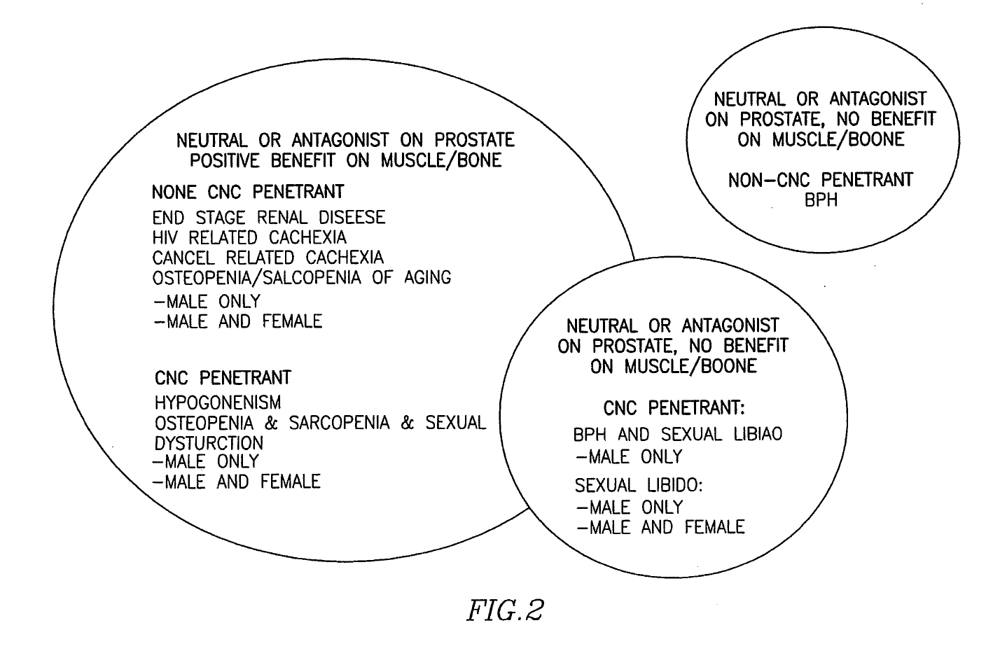

As shown in FIGS. 1 and 2, one subgroup of SARM compounds have no effect on muscle and bone, and have neutral or antagonistic effect on prostate. Within this subgroup, those SARM compounds that do not penetrate the CNS are effective at treating or preventing benign prostate hyperplasia (BPH). Those SARM compounds that are able to penetrate the CNS are effective at treating or preventing sexual dysfunction.

Furthermore, as shown in FIGS. 1 and 2, another subgroup of SARM compounds have an agonistic activity on muscle and bone, and have neutral or antagonistic effect on prostate. Within this subgroup, those SARM compounds that do not penetrate the CNS are effective at treating or preventing sarcopenia and osteopenia. Those SARM compounds that are able to penetrate the CNS are effective at treating or preventing hypogonadism, sexual dysfunction, sarcopenia and osteopenia.

The SARM compounds of the present invention are a novel class of androgen receptor targeting agents (ARTA) which demonstrate androgenic or antiandrogenic and anabolic activity of a nonsteroidal ligand for the androgen receptor. The agents define a new subclass of compounds, which are selective androgen receptor modulators (SARMs).

The androgen receptor (AR) is a ligand-activated transcriptional regulatory protein that mediates induction of male sexual development and function through its activity with endogenous androgens (male sex hormones). The androgenic hormones are steroids which are produced in the body by the testis and the cortex of the adrenal gland. Androgenic steroids play an important role in many physiologic processes, including the development and maintenance of male sexual characteristics such as muscle and bone mass, prostate growth, spermatogenesis, and the male hair pattern (Matsumoto, Endocrinol. Met. Clin. N. Am. 23:857-75 (1994)). The endogenous steroidal androgens include testosterone and dihydrotestosterone (“DHT”). Other steroidal androgens include esters of testosterone, such as the cypionate, propionate, phenylpropionate, cyclopentylpropionate, isocarporate, enanthate, and decanoate esters, and other synthetic andiogens such as 7-Methyl-Nortestosterone (“MENT”) and its acetate ester (Sundaram et al., “7 Alpha-Methyl-Nortestosterone (MENT): The Optimal Androgen For Male Contraception,” Ann. Med., 25:199-205 (1993) (“Sundaram”)).

As contemplated herein, this invention provides a class of compounds which are Selective Androgen Receptor Modulator (SARM) compounds. These compounds, which are useful in preventing and treating ADAM-associated conditions are classified as androgen receptor agonists (AR agonists), partial agonists or androgen receptor antagonists (AR antagonists).

A receptor agonist is a substance which binds receptors and activates them. A receptor partial agonist is a substance which binds receptor and partially activate them. A receptor antagonist is a substance which binds receptors and inactivates them. As demonstrated herein, the SARM compounds of the present invention have a tissue-selective effect, wherein one agent may be an agonist, partial agonist and/or antagonist, depending on the tissue. For example, the SARM compound may stimulate muscle tissue and at the same time inhibit prostate tissue. In one embodiment, the SARMs which are useful in treating and preventing ADAM-associated conditions are AR agonists, and are, therefore, useful in binding to and activating the AR. In another embodiment, the SARMs which are useful in treating and preventing ADAM-associated conditions are AR antagonists, and are, therefore, useful in binding to and inactivating the AR. Assays to determine whether the compounds of the present invention are AR agonists or antagonists are well known to a person skilled in the art. For example, AR agonistic activity can be determined by monitoring the ability of the SARM compounds to maintain and/or stimulate the growth of AR containing tissue such as prostate and seminal vesicles, as measured by weight. AR antagonistic activity can be determined by monitoring the ability of the SARM compounds inhibit the growth of AR containing tissue.

In yet another embodiment, the SARM compounds of the present invention can be classified as partial AR agonist/antagonists. The SARMs are AR agonists in some tissues, to cause increased transcription of AR-responsive genes (e.g. muscle anabolic effect). In other tissues, these compounds serve as competitive inhibitors of testosterone/DHT on the AR to prevent agonistic effects of the native androgens.

The SARM compounds of the present invention bind either reversibly or irreversibly to the androgen receptor. In one embodiment, the SARM compounds bind reversibly to the androgen receptor. In another embodiment, the SARM compounds bind irreversibly to the androgen receptor. The compounds of the present invention may contain a functional group (affinity label) that allows alkylation of the androgen receptor (i.e. covalent bond formation). Thus, in this case, the compounds bind irreversibly to the receptor and, accordingly, cannot be displaced by a steroid, such as the endogenous ligands DHT and testosterone.

Pharmaceutical Compositions

The treatment methods of the present invention comprise, in one embodiment, administering a pharmaceutical preparation comprising the SARM compound and/or its analog, derivative, isomer, metabolite, pharmaceutically acceptable salt, pharmaceutical product, hydrate, N-oxide, prodrug, polymorph, crystal or any combination thereof, and a pharmaceutically acceptable carrier.

As used herein, “pharmaceutical composition” means a composition comprising an “effective amount” of the active ingredient, i.e. the SARM compound, together with a pharmaceutically acceptable carrier or diluent.

An “effective amount” as used herein refers to that amount which provides a therapeutic effect for a given condition and administration regimen. An “effective amount” of the SARM compounds as used herein can be in the range of 1-500 mg/day. In one embodiment the dosage is in the range of 1-100 mg/day. In another embodiment the dosage is in the range of 100-500 mg/day. In another embodiment the dosage is in a range of 45-60 mg/day. In another embodiment the dosage is in the range of 15-25 mg/day. In another embodiment the dosage is in the range of 55-65 mg/day. In another embodiment the dosage is in the range of 45-60 mg/day. The SARM compounds can be administered daily, in single dosage forms containing the entire amount of daily dose, or can be administered daily in multiple doses such as twice daily or three times daily. The SARM compounds can also be administered intermittently, for example every other day, 3 days a week, four days a week, five days a week and the like.

As used herein, the term “treating” includes preventative as well as disorder remitative treatment. As used herein, the terms “reducing”, “suppressing” and “inhibiting” have their commonly understood meaning of lessening or decreasing. As used herein, the term “facilitating” is giving its commonly understood meaning of increasing the rate. As used herein, the term “promoting” is given its commonly understood meaning of increasing. As used herein, the term “progression” means increasing in scope or severity, advancing, growing or becoming worse.

As used herein, the term “administering” refers to bringing a subject in contact with a SARM compound of the present invention. As used herein, administration can be accomplished in vitro, i.e. in a test tube, or in vivo, i.e. in cells or tissues of living organisms, for example humans. In one embodiment, the present invention encompasses administering the compounds of the present invention to a subject. In one embodiment, the subject is a mammalian subject. In another embodiment, the subject is a human.

The pharmaceutical compositions containing the SARM agent can be administered to a subject by any method known to a person skilled in the art, such as parenterally, paracancerally, transmucosally, transdermally, intramuscularly, intravenously, intradermally, subcutaneously, intraperitonealy, intraventricularly, intracranially, intravaginally or intratumorally.

In one embodiment, the pharmaceutical compositions are administered orally, and are thus formulated in a form suitable for oral administration, i.e. as a solid or a liquid preparation. Suitable solid oral formulations include tablets, capsules, pills, granules, pellets and the like. Suitable liquid oral formulations include solutions, suspensions, dispersions, emulstions, oils and the like. In one embodiment of the present invention, the SARM compounds are formulated in a capsule. In accordance with this embodiment, the compositions of the present invention comprise in addition to the SARM active compound and the inert carrier or diluent, a hard gelating capsule.

Further, in another embodiment, the pharmaceutical compositions are administered by intravenous, intraarterial, or intramuscular injection of a liquid preparation. Suitable liquid formulations include solutions, suspensions, dispersions, emulsions, oils and the like. In one embodiment, the pharmaceutical compositions are administered intravenously, and are thus formulated in a form suitable for intravenous administration. In another embodiment, the pharmaceutical compositions are administered intraarterially, and are thus formulated in a form suitable for intraarterial administration. In another embodiment, the pharmaceutical compositions are administered intramuscularly, and are thus formulated in a form suitable for intramuscular administration.

Further, in another embodiment, the pharmaceutical compositions are administered topically to body surfaces, and are thus formulated in a form suitable for topical administration. Suitable topical formulations include gels, ointments, creams, lotions, drops and the like. For topical administration, the SARM agents or their physiologically tolerated derivatives such as salts, esters, N-oxides, and the like are prepared and applied as solutions, suspensions, or emulsions in a physiologically acceptable diluent with or without a pharmaceutical carrier.

Further, in another embodiment, the pharmaceutical compositions are administered as a suppository, for example a rectal suppository or a urethral suppository. Further, in another embodiment, the pharmaceutical compositions are administered by subcutaneous implantation of a pellet. In a further embodiment, the pellet provides for controlled release of SARM agent over a period of time.

In another embodiment, the active compound can be delivered in a vesicle, in particular a liposome (see Langer, Science 249:1527-1533 (1990); Treat et al., in Liposomes in the Therapy of Infectious Disease and Cancer, Lopez-Berestein and Fidler (eds.), Liss, New York, pp. 353-365 (1989); Lopez-Berestein, ibid., pp 317-327; see generally ibid).

As used herein “pharmaceutically acceptable carriers or diluents” are well known to those skilled in the art. The carrier or diluent may be a solid carrier or diluent for solid formuations, a liquid carrier or diluent for liquid formulations, or mixtures thereof.

Solid carriers/diluents include, but are not limited to, a gum, a starch (e.g. corn starch, pregeletanized starch), a sugar (e.g., lactose, mannitol, sucrose, dextrose), a cellulosic material (e.g. microcrystalline cellulose), an acrylate (e.g. polymethylacrylate), calcium carbonate, magnesium oxide, talc, or mixtures thereof.

For liquid formulations, pharmaceutically acceptable carriers may be aqueous or non-aqueous solutions, suspensions, emulsions or oils. Examples of non-aqueous solvents are propylene glycol, polyethylene glycol, and injectable organic esters such as ethyl oleate. Aqueous carriers include water, alcoholic/aqueous solutions, emulsions or suspensions, including saline and buffered media. Examples of oils are those of petroleum, animal, vegetable, or synthetic origin, for example, peanut oil, soybean oil, mineral oil, olive oil, sunflower oil, and fish-liver oil.

Parenteral vehicles (for subcutaneous, intravenous, intraarterial, or intramuscular injection) include sodium chloride solution, Ringer's dextrose, dextrose and sodium chloride, lactated Ringer's and fixed oils. Intravenous vehicles include fluid and nutrient replenishers, electrolyte replenishers such as those based on Ringer's dextrose, and the like. Examples are sterile liquids such as water and oils, with or without the addition of a surfactant and other pharmaceutically acceptable adjuvants. In general, water, saline, aqueous dextrose and related sugar solutions, and glycols such as propylene glycols or polyethylene glycol are preferred liquid carriers, particularly for injectable solutions, Examples of oils are those of petroleum, animal, vegetable, or synthetic origin, for example, peanut oil, soybean oil, mineral oil, olive oil, sunflower oil, and fish-liver oil.

In addition, the compositions may further comprise binders (e.g. acacia, cornstarch, gelatin, carbomer, ethyl cellulose, guar gum, hydroxypropyl cellulose, hydroxypropyl methyl cellulose, povidone), disintegrating agents (e.g. cornstarch, potato starch, alginic acid, silicon dioxide, croscarmelose sodium, crospovidone, guar gum, sodium starch glycolate), buffers (e.g., Tris-HCl., acetate, phosphate) of various pH and ionic strength, additives such as albumin or gelatin to prevent absorption to surfaces, detergents (e.g., Tween 20, Tween 80, Pluronic F68, bile acid salts), protease inhibitors, surfactants (e.g. sodium lauryl sulfate), permeation enhancers, solubilizing agents (e.g., glycerol, polyethylene glycerol), anti-oxidants (e.g., ascorbic acid, sodium metabisulfite, butylated hydroxyanisole), stabilizers (e.g. hydroxypropyl cellulose, hyroxypropylmethyl cellulose), viscosity increasing agents (e.g. carbomer, colloidal silicon dioxide, ethyl cellulose, guar gum), sweetners (e.g. aspartame, citric acid), preservatives (e.g., Thimerosal, benzyl alcohol, parabens), lubricants (e.g. stearic acid, magnesium stearate, polyethylene glycol, sodium lauryl sulfate), flow-aids (e.g. colloidal silicon dioxide), plasticizers (e.g. diethyl phthalate, triethyl citrate), emulsifiers (e.g. carbomer, hydroxypropyl cellulose, sodium lauryl sulfate), polymer coatings (e.g., poloxamers or poloxamines), coating and film forming agents (e.g. ethyl cellulose, acrylates, polymethacrylates) and/or adjuvants.

In one embodiment, the pharmaceutical compositions provided herein are controlled release compositions, i.e. compositions in which the SARM compound is released over a period of time after administration. Controlled or sustained release compositions include formulation in lipophilic depots (e.g. fatty acids, waxes, oils). In another embodiment, the composition is an immediate release composition, i.e. a composition in which all of the SARM compound is released immediately after administration.

In yet another embodiment, the pharmaceutical composition can be delivered in a controlled release system. For example, the agent may be administered using intravenous infusion, an implantable osmotic pump, a transdermal patch, liposomes, or other modes of administration. In one embodiment, a pump may be used (see Langer, supra; Sefton, CRC Crit. Ref. Biomed. Eng. 14:201 (1987); Buchwald et al., Surgery 88:507 (1980); Saudek et al., N. Engl. J. Med. 321:574 (1989). In another embodiment, polymeric materials can be used. In yet another embodiment, a controlled release system can be placed in proximity to the therapeutic target, i.e., the brain, thus requiring only a fraction of the systemic dose (see, e.g., Goodson, in Medical Applications of Controlled Release, supra, vol. 2, pp. 115-138 (1984). Other controlled release systems are discussed in the review by Langer (Science 249:1527-1533 (1990).

The compositions may also include incorporation of the active material into or onto particulate preparations of polymeric compounds such as polylactic acid, polglycolic acid, hydrogels, etc, or onto liposomes, microemulsions, micelles, unilamellar or multilamellar vesicles, erythrocyte ghosts, or spheroplasts.) Such compositions will influence the physical state, solubility, stability, rate of in vivo release, and rate of in vivo clearance.

Also comprehended by the invention are particulate compositions coated with polymers (e.g. poloxamers or poloxamines) and the compound coupled to antibodies directed against tissue-specific receptors, ligands or antigens or coupled to ligands of tissue-specific receptors.

Also comprehended by the invention are compounds modified by the covalent attachment of water-soluble polymers such as polyethylene glycol, copolymers of polyethylene glycol and polypropylene glycol, carboxymethyl cellulose, dextran, polyvinyl alcohol, polyvinylpyrrolidone or polyproline. The modified compounds are known to exhibit substantially longer half-lives in blood following intravenous injection than do the corresponding unmodified compounds (Abuchowski et al., 1981; Newmark et al., 1982; and Katre et al., 1987). Such modifications may also increase the compound's solubility in aqueous solution, eliminate aggregation, enhance the physical and chemical stability of the compound, and greatly reduce the immunogenicity and reactivity of the compound. As a result, the desired in vivo biological activity may be achieved by the administration of such polymer-compound abducts less frequently or in lower doses than with the unmodified compound.

The preparation of pharmaceutical compositions which contain an active component is well understood in the art, for example by mixing, granulating, or tablet-forming processes. The active therapeutic ingredient is often mixed with excipients which are pharmaceutically acceptable and compatible with the active ingredient. For oral administration, the SARM agents or their physiologically tolerated derivatives such as salts, esters, N-oxides, and the like are mixed with additives customary for this purpose, such as vehicles, stabilizers, or inert diluents, and converted by customary methods into suitable forms for administration, such as tablets, coated tablets, hard or soft gelatin capsules, aqueous, alcoholic or oily solutions. For parenteral administration, the SARM agents or their physiologically tolerated derivatives such as salts, esters, N-oxides, and the like are converted into a solution, suspension, or emulsion, if desired with the substances customary and suitable for this purpose, for example, solubilizers or other.

An active component can be formulated into the composition as neutralized pharmaceutically acceptable salt forms. Pharmaceutically acceptable salts include the acid addition salts (formed with the free amino groups of the polypeptide or antibody molecule), which are formed with inorganic acids such as, for example, hydrochloric or phosphoric acids, or such organic acids as acetic, oxalic, tartaric, mandelic, and the like.

Salts formed from the free carboxyl groups can also be derived from inorganic bases such as, for example, sodium, potassium, ammonium, calcium, or ferric hydroxides, and such organic bases as isopropylamine, trimethylamine, 2-ethylamino ethanol, histidine, procaine, and the like.

For use in medicine, the salts of the SARM will be pharmaceutically acceptable salts. Other salts may, however, be useful in the preparation of the compounds according to the invention or of their pharmaceutically acceptable salts. Suitable pharmaceutically acceptable salts of the compounds of this invention include acid addition salts which may, for example, be formed by mixing a solution of the compound according to the invention with a solution of a pharmaceutically acceptable acid such as hydrochloric acid, sulphuric acid, methanesulphonic acid, fumaric acid, maleic acid, succinic acid, acetic acid, benzoic: acid, oxalic acid, citric acid, tartaric acid, carbonic acid or phosphoric acid.

In one embodiment, the methods of the present invention comprise administering a SARM compound as the sole active ingredient. However, also encompassed within the scope of the present invention are methods for treating and/or preventing ADAM-associated conditions as described herein, which comprise administering the SARM compounds in combination with one or more therapeutic agents. These agents include, but are not limited to: LHRH analogs, reversible antiandrogens, antiestrogens, anticancer drugs, 5-alpha reductase inhibitors, aromatase inhibitors, progestins, or agents acting through other nuclear hormone receptors.

Thus, in one embodiment, the present invention provides compositions and pharmaceutical compositions comprising a selective androgen receptor modulator compound, in combination with an LHRH analog. In another embodiment, the present invention provides compositions and pharmaceutical compositions comprising a selective androgen receptor modulator compound, in combination with a reversible antiandrogen. In another embodiment, the present invention provides compositions and pharmaceutical compositions comprising a selective androgen receptor modulator compound, in combination with an antiestrogen. In another embodiment, the present invention provides compositions and pharmaceutical compositions comprising a selective androgen receptor modulator compound, in combination with an anticancer drug. In another embodiment, the present invention provides compositions and pharmaceutical compositions comprising a selective androgen receptor modulator compound, in combination with a 5-alpha reductase inhibitor. In another embodiment, the present invention provides compositions and pharmaceutical compositions comprising a selective androgen receptor modulator compound, in combination with an aromatase inhibitor. In another embodiment, the present invention provides compositions and pharmaceutical compositions comprising a selective androgen receptor modulator compound, in combination with a progestin. In another embodiment, the present invention provides compositions and pharmaceutical compositions comprising a selective androgen receptor modulator compound, in combination with an agent acting through other nuclear hormone receptors.

The following examples are presented in order to more fully illustrate the preferred embodiments of the invention. They should in no way, however, be construed as limiting the broad scope of the invention.

### EXPERIMENTAL DETAILS SECTION

### Example 1

### Pharmacologic Activity and Tissue-Selectivity of Compound VI and Compound VII in Rats of Varying Hormonal Status

Selective androgen receptor modulators (SARMs) have a wide variety of potential therapeutic applications, including male hypogonadism, osteoporosis, muscle-wasting diseases, sexual libido and contraception. Previous studies by Applicants demonstrated that Compound VI is a potent and efficacious selective androgen receptor modulator (SARM) in castrated male rats. To provide a representative model of the vast majority of men that will eventually receive this drug, Applicants completed a preclinical study to compare the pharmacologic effects and tissue-selectivity of Compound VI, Compound VII—another potent SARM, and testosterone propionate (TP) in male rats of varying hormonal status. Male rats with normal testicular function (i.e., intact with no surgical manipulation) were included to examine the effects of Compound VI and Compound VII on animals with normal blood levels of testosterone. Male rats that received unilateral orchidectomy (i.e., surgical removal of one testis) were included to examine the effects of Compound VI and Compound VII on animals with slight androgen depletion. Male rats that received bilateral orchidectomy (i.e., surgical removal of both testes) were included to examine the effects of Compound VI and Compound VII on androgen-deficient animals. 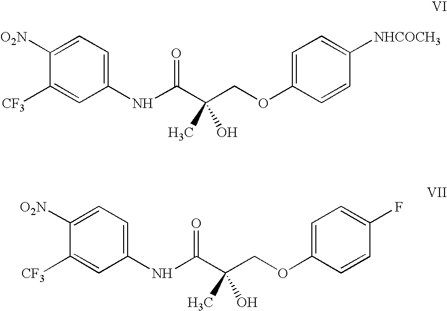
Methods:

Compound VI and Compound VII were synthesized and characterized in the laboratory of Dr. Duane Miller at the University of Tennessee, Memphis, Tenn. Male Sprague-Dawley rats were purchased from Harlan Biosciences (Indianapolis, Ind.). The animals were maintained on a 12-h cycle of light and dark with food and water available ad libitum. All animal studies were reviewed and approved by the Animal Care and Use Committee of The Ohio State University, and conformed to the Principles of Laboratory Animal Care (NIH publication #85-23, revised 1985). Immature male Sprague-Dawley rats weighing 187 to 214 g were randomly distributed into 9 groups of 5 animals. One day before the initiation of drug treatment, groups 4 through 6 and groups 7 through 9 received unilateral or bilateral orchidectomy, respectively, via a midline scrotal incision. Groups 1 through 3 did not undergo surgery. All drugs given to animals were freshly prepared as solutions in polyethylene glycol 300 (PEG 300). Groups 4 and 7 received treatment with vehicle alone (i.e., PEG 300). Animals in groups 3, 6, and 9 received testosterone propionate (TP, 0.5 mg/day) via implantation of subdermal osmotic pumps (Model 2002, Durect Corporation, Palo Alto, Calif.). Animals in groups 2, 5, and 8 received Compound VI or Compound VII (0.5 mg/day) via implantation of subdermal osmotic pumps. After 14 days of drug treatment, rats were weighed, anesthetized, and sacrificed. Blood samples were collected by venipuncture of the abdominal aorta. Plasma samples were analyzed for testosterone, FSH, LH and osteocalcin. Testosterone concentrations were measured by AniLytics Inc. (Gaithersburg, Md.). FSH and LH levels were measured by the National Hormone and Peptide Program (Dr. A F Parlow, UCLA, CA). Plasma osteocalcin levels were determined using a commercially available rat osteocalcin EIA kit from Biomedical Technologies Inc. (Stoughton, Mass.). The ventral prostates, seminal vesicles, and levator ani muscle were removed and weighed. Osmotic pumps were also removed from animals to check for correct pump operation. The weights of all organs were normalized to body weight, and analyzed for any statistically significant differences between groups using single-factor ANOVA with the alpha value set a priori at p<0.05. The weights of prostates and seminal vesicles were used as indices for evaluation of androgenic activity, and the levator ani muscle weight was used to evaluate the anabolic activity. Statistical analyses of parameters from complete blood count or serum chemical profiling, wherever applicable, were performed by single-factor ANOVA with the alpha value set a priori at p<0.05.

Results:

Plasma testosterone levels were significantly lower in castrated rats, regardless of the treatment group (Table 1). Unilateral orchidectomy led to a slight but statistically insignificant decrease in plasma testosterone concentrations. Castrated male rats that received exogenous TP (0.5 mg/day) had higher plasma testosterone levels than vehicle-treated and Compound VI treated controls. However, there were no significant differences in plasma testosterone levels between hemi-orchidectomized animals in any of the treatment groups. Compound VI treatment did not affect testosterone levels in intact, hemi-orchidectomized or castrated male rats, demonstrating that Compound VI has little to no effect on endogenous androgen production at pharmacologically relevant doses.

TABLE 1
Plasma testosterone levels (ng/ml) in different treatment groups
(n = 5).

| | | Compound VI | |
|---|---|---|---|
| | Control | (0.5 mg/day) | TP (0.5 mg/day) |
| Intact | 2.674 ± 1.476 | 1.830 ± 0.510 | 1.482 ± 0.416 |
| Hemi-orchidectomized | 1.740 ± 1.049 | 1.404 ± 0.810 | 2.366 ± 1.232 |
| Castrated | 0.036 ± 0.075^{†‡} | 0.066 ± 0.148^{†‡} | 0.258 ± 0.103*^{†‡} |

*p < 0.05 compared to control group.
^{†}p < 0.05 compared to intact group.
^{‡}p < 0.05 compared to hemi-orchidectomized group.

Plasma FSH and LH levels (Tables 2 and 3) significantly increased in animals that received bilateral orchidectomy (i.e., castrated controls). Plasma FSH levels and LH levels in hemi-orchidectomized animals were not significantly different than intact animals, corroborating the observation that unilateral orchidectomy had no effect on plasma testosterone levels or the pituitary hormones that regulate it. Treatment with TP caused a significant decrease in FSH and LH levels in castrated male rats, indicating that TP suppresses pituitary hormone production. However, Compound VI had no effect on plasma FSH and LH levels. These data indicate that Compound VI has no effect on pituitary hormone production and is therefore advantageous to TP for use in intact animals. No significant differences in FSH or LH levels were observed in intact or hemi-orchidectomized animals.

TABLE 2
Plasma FSH levels (ng/ml) in different treatment groups (n = 5).

| | | Compound VI | |
|---|---|---|---|
| | Control | (0.5 mg/day) | TP (0.5 mg/day) |
| Intact | 13.0 ± 1.3 | 14.4 ± 1.7 | 11.4 ± 1.7 |
| Hemi- | 18.0 ± 1.9^{†} | 15.2 ± 2.2 | 17.2 ± 3.3^{†} |
| orchidectomized | | | |
| Castrated | 68.6 ± 6.3^{†‡} | 69.6 ± 11.7^{†‡} | 58.0 ± 6.9*^{†‡} |

*p < 0.05 compared to control group.
^{†}p < 0.05 compared to intact group.
^{‡}p < 0.05 compared to hemi-orchidectomized group.

TABLE 3
Plasma LH levels (ng/ml) in different treatment groups (n = 5).

| | | Compound VI | |
|---|---|---|---|
| | Control | (0.5 mg/day) | TP (0.5 mg/day) |
| Intact | 0.160 ± 0.187 | 0.026 ± 0.037 | 0.168 ± 0.173 |
| Hemi-orchidectomized | 0.240 ± 0.268 | 0.124 ± 0.115 | 0.124 ± 0.092 |
| Castrated | 8.704 ± 1.709^{†‡} | 8.644 ± 2.799^{†‡} | 6.702 ± 1.513^{†‡} |

*p < 0.05 compared to control group.
^{†}p < 0.05 compared to intact group.
^{‡}p < 0.05 compared to hemi-orchidectomized group.

Applicants also examined the effects of unilateral orchidectomy, bilateral orchidectomy, TP, and Compound VI on plasma osteocalcin levels (Table 4). Osteocalcin is a specific osteoblastic marker that can be used to evaluate the endogenous bone formation rate. There were no significant differences in osteocalcin levels between intact, hemi-orchidectomized and castrated animals in the vehicle-treated (i.e., control) animals. However, treatment with Compound VI led to a significant increase in plasma osteocalcin levels in hemi-orchidectomized and castrated animals. TP had no effect on plasma osteocalcin levels. These data suggest that Compound VI increases bone formation rate in male animals with no effects on plasma concentrations of testosterone, FSH, or LH. These data corroborate our findings in pilot studies examining bone mineral content and bone mineral density in gonadectomized male and female animals.

TABLE 4
Plasma osteocalcin levels (ng/ml) in different treatment groups
(n = 5).

| | | Compound VI | |
|---|---|---|---|
| | Control | (0.5 mg/day) | TP (0.5 mg/day) |
| Intact | 59.403 ± 13.933 | 55.584 ± 9.715 | 74.952 ± 15.399 |
| Hemi-orchidectomized | 62.110 ± 14.770 | 89.804 ± 15.517*^{†} | 77.236 ± 24.418 |
| Castrated | 66.965 ± 11.305 | 94.215 ± 12.568*^{†} | 65.976 ± 11.213 |

*p < 0.05 compared to control group.
^{†}p < 0.05 compared to intact group.
^{‡}p < 0.05 compared to hemi-orchidectomized group.

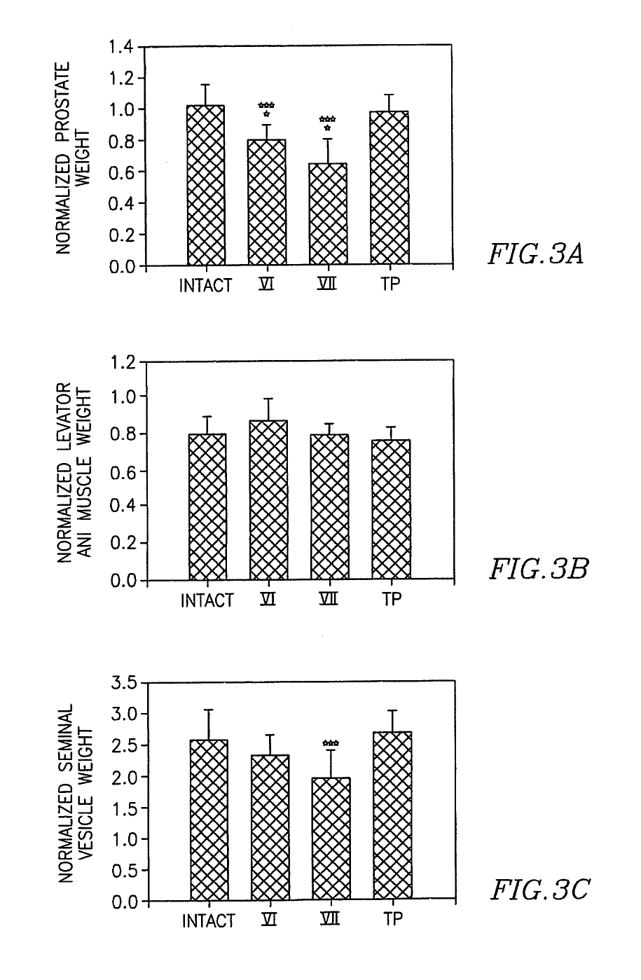

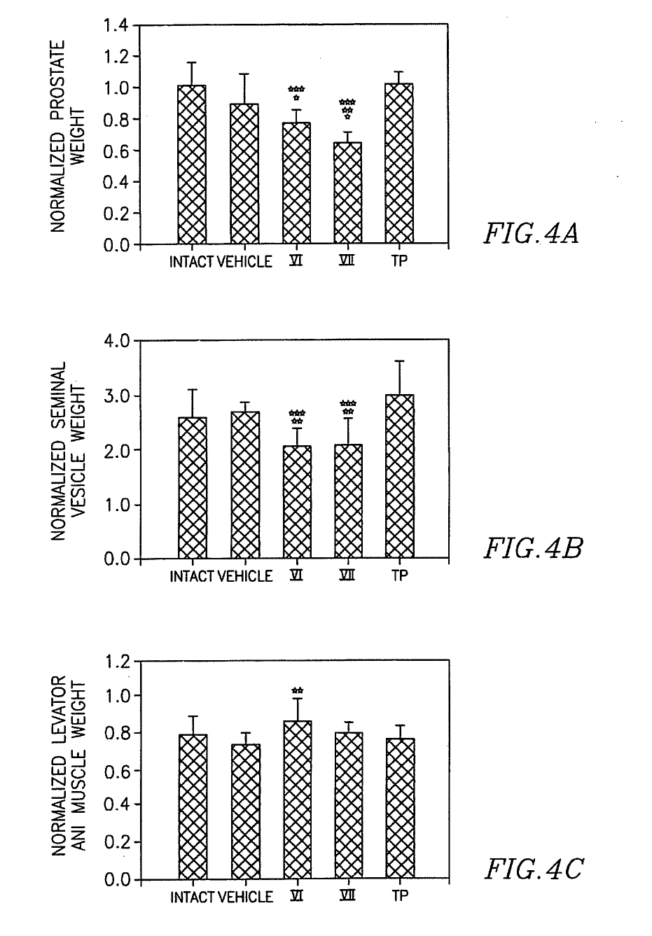

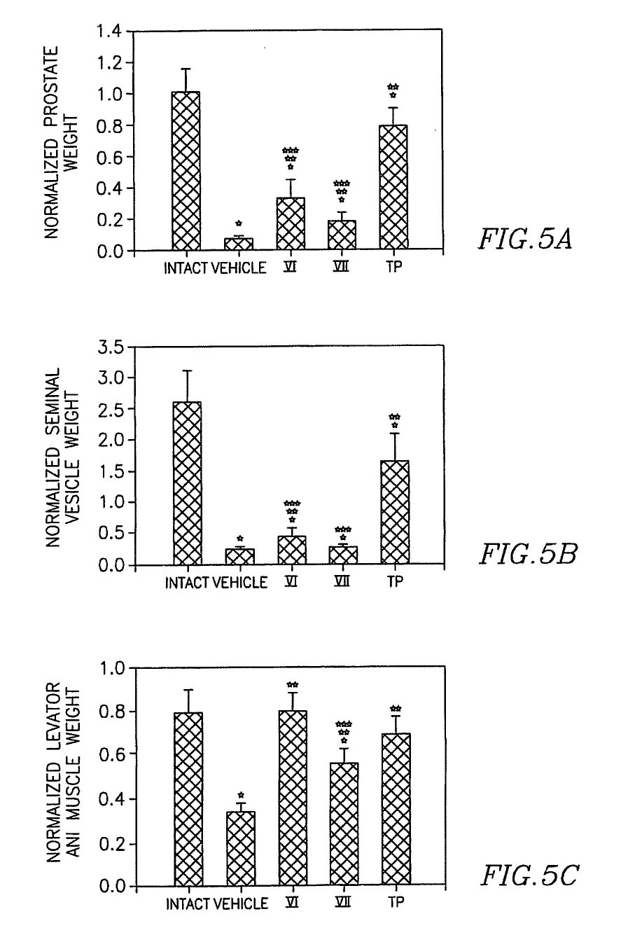

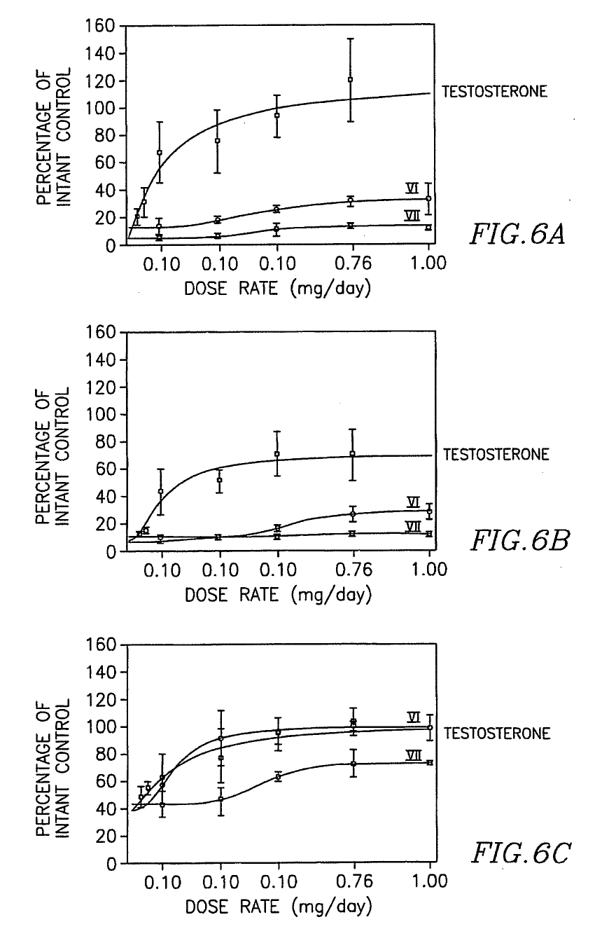

As shown in Table 5 and FIG. 3, in intact animals, Compound VI decreased the size of the prostate to 79% and, of that observed in control animals (FIG. 3A), with no statistically significant changes in the size of the seminal vesicles (FIG. 3B) or levator ani muscle (FIG. 3C). The pharmacologic effects and tissue selectivity of Compound VI were more obvious in hemi-orchidectomized animals (Table 5 and FIG. 4). Compound VI decreased the size of the prostate (FIG. 4A) and seminal vesicles (FIG. 4B) to 75% and 79%, respectively, and increased the size of the levator ani muscle (FIG. 4C) to 108% of that observed in untreated hemi-orchidectomized animals. These observations demonstrate that Compound VI acts as a partial agonist in prostate and seminal vesicles and as a full agonist in levator ani muscle. No adverse pharmacologic effects were observed. Similarly, as shown in Table 5 and in FIGS. 5 and 6, in castrated animals,

TABLE 5
Comparison of androgenic and anabolic effects of Compound VI
and TP on intact, hemi-orchidectomized and castrated rats
(% of intact control, n = 5).

| | | | Compound VI | TP |
|---|---|---|---|---|
| Organs | | Control | (0.5 mg/day) | (0.5 mg/day) |
| Prostate | Intact | 100.00 ± 13.13 | 79.41 ± | 97.45 ± 10.82 |
| | | | 9.32*^{†} | |
| | Hemi- | 86.42 ± 19.52 | 74.69 ± | 98.57 ± 7.98 |
| | | | 8.44*^{†} | |
| | Castrated | 7.19 ± 1.25 | 32.55 ± | 76.78 ± 10.43*^{‡} |
| | | | 11.65*^{†‡} | |
| Seminal | Intact | 100.00 ± 18.84 | 90.54 ± | 103.95 ± 13.23 |
| Vesicle | | | 12.10 | |
| | Hemi- | 102.93 ± 7.47 | 78.55 ± | 114.19 ± 23.81 |
| | | | 13.58^{†‡} | |
| | Castrated | 8.97 ± 1.23 | 16.47 ± | 63.48 ± 17.05*^{‡} |
| | | | 5.21*^{†‡} | |
| Levator | Intact | 100.00 ± 12.69 | 109.15 ± | 95.61 ± 9.34 |
| Ani | | | 14.68 | |
| | Hemi- | 92.94 ± 7.83 | 108.10 ± | 98.63 ± 10.47 |
| | | | 8.92^{‡} | |
| | Castrated | 42.74 ± 5.22 | 100.65 ± | 87.27 ± 10.25^{‡} |
| | | | 10.86^{‡} | |

*p < 0.05 compared to intact control group.
^{†}p < 0.05 compared to TP of same surgical status (i.e., intact, hemi-orchidectomized, or castrate).
^{‡}p < 0.05 compared to control group of same surgical status.

A comparison of the androgenic and anabolic activities of Compound VII and Compound VI is provided in Table 6.

TABLE 2
Comparison of Androgenic and Anabolic Activities of
GTx-014 and GTx-017 to TP

| | | | Eₘₐₓ | Relative | ED₅₀ | Relative |
|---|---|---|---|---|---|---|
| | Organs | Treatment | (% of Intact Control) | Afficacy | (mg/day) | Potency |
| Androgenic | | TP | 120.6 ± 13.4 | 1.00 | 0.13 ± 0.03 | 1.00 |
| | Prostate | GTx-014 | 14.5 ± 0.7 | 0.12 | 0.42 ± 0.04 | 0.31 |
| | | GTx-017 | 35.2 ± 0.4 | 0.29 | 0.43 ± 0.01 | 0.30 |
| | Seminal | TP | 70.0 ± 18.8 | 1.00 | 0.12 ± 0.02 | 1.00 |
| | Vesicle | GTx-014 | 12.7 ± 3.1 | 0.18 | 0.38 ± 0.26 | 0.32 |
| | | GTx-017 | 28.5 ± 0.8 | 0.40 | 0.55 ± 0.02 | 0.22 |
| Anabolic | Levator | TP | 104.2 ± 10.1 | 1.00 | 0.15 ± 0.03 | 1.00 |
| | Ani | GTx-014 | 74.9 ± 0.4 | 0.72 | 0.44 ± 0.01 | 0.34 |
| | Muscle | GTx-017 | 101.0 ± 1.0 | 0.97 | 0.14 ± 0.01 | 1.07 |

Conclusions:

Compound VI demonstrated potent and tissue-selective pharmacologic effects in intact, hemi-orchidectomized and castrated male rats. Compound VI led to significant decreases in prostate weights in intact and hemi-orchidectomized animals, and was less effective than TP at increasing the weight of the prostate in castrated animals. Similar pharmacologic effects were noted in the seminal vesicles (another organ generally considered as a marker of androgenic effects), with the exception that Compound VI had no effect on the weight of the seminal vesicles in intact animals. Compound VI treatment led to significant increases in the weight of the levator ani muscle in hemi-orchidectomized and castrated animals. These effects were greater than those observed with TP. These data demonstrate the tissue-selective pharmacologic effects of Compound VI. It is important to note that these effects were observed in the absence of any significant changes in plasma concentrations of FSH, LH and testosterone. Compound VI increased plasma concentrations of osteocalcin. In summary, these data show that Compound VI elicits an optimal pharmacological profile in male animals, identifying it as the first member of a new class of orally bioavailable and tissue-selective SARMs.

### Example 2

### Effect of Compound VI on Myosin Heavy Chain (MHC) Subtype IIb m-RNA Expression

To further demonstrate the importance of Compound VI in muscle, Applicants have examined the effects of this nonsteroidal anabolic agent directly in skeletal muscle by monitoring the expression of myosin heavy chain (MHC) subtypes using RT-PCR. MHC is the predominant protein in skeletal muscle encoded by a multigene family expressed in a tissue-specific and developmentally regulated manner [Adams et al 1999]. In steady state, mRNA expression usually parallels the pattern of MHC protein expression. Because transcription of MHC mRNA occurs in advance of MHC protein translation, and the increased sensitivity of RT-PCR compared to western blotting, rapid changes in mRNA expression can be detected and used to analyze the subtle dynamic effects of muscle anabolism [Wright et al 1997].

Results:

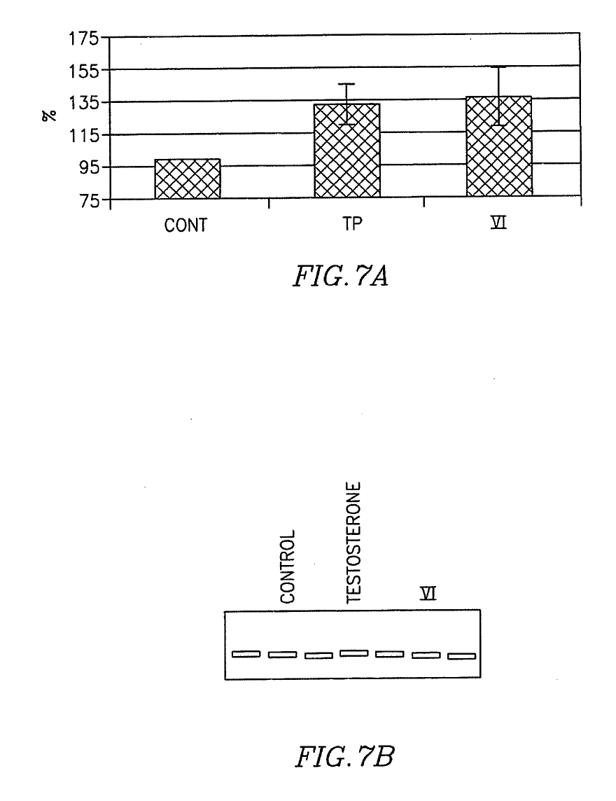

The masseter muscle dissected from untreated intact female rats was set as the control level (representing 100%) of MHC IIb expression (FIG. 7A). Intact female rats treated with androgens were evaluated against the untreated controls for the effect of treatment on MHC IIb from masseter. The results indicate that testosterone propionate has a positive effect on masseter muscle where it increased transcription of MHC type IIb to 133% of untreated control (FIG. 7A). Compound VI was also anabolic in muscle, with an increase in MHC type IIb to 137% (FIG. 7A). Actual untransformed PCR data is shown in FIG. 7B.

### Effect of SAMS on Bone Resorption in Rats

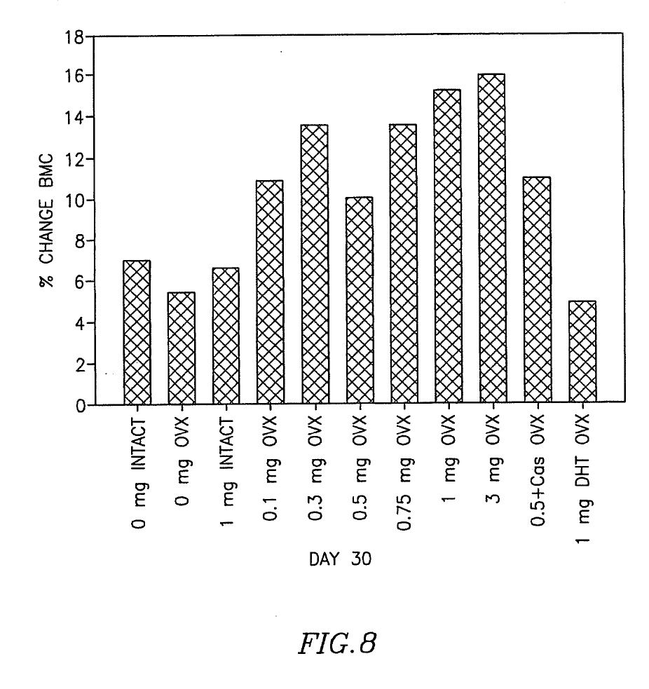

One hundred ten female rats were assigned to one of eleven treatment groups. Groups 1-8 were ovariectomized on day one of the study. Groups 9-11 were intact animals. Groups 1-6 received Compound VI by daily subcutaneous injection at doses of 0.1, 0.3, 0.5, 0.75, 1.0, and 3 mg/day, respectively. Groups 7 and 11 received dihydrotestosterone (DHT) at a dose of 1 mg/day. Groups 8 and 9 were ovariectomized and intact control groups, respectively. Group 10 received Compound VI at a dose of 1.0 mg/day. All animals were treated for 120 days. Bone mineral content (BMC) was determined using dual energy x-ray absorptiometry (DEXA) on days 1, 30, 60, 90, and 120. See FIG. 8.

A time and dose-dependent increase in BMC for all of Compound VI treated groups, with increases of 22.9, 26.0, 28.5, 30.5, 30.0, and 40.1% observed in groups 1-6, respectively. DHT increased BMC by 15% at a dose of 1 mg/day. Compound VI inhibited bone resorption and was more potent than DHT in this model.

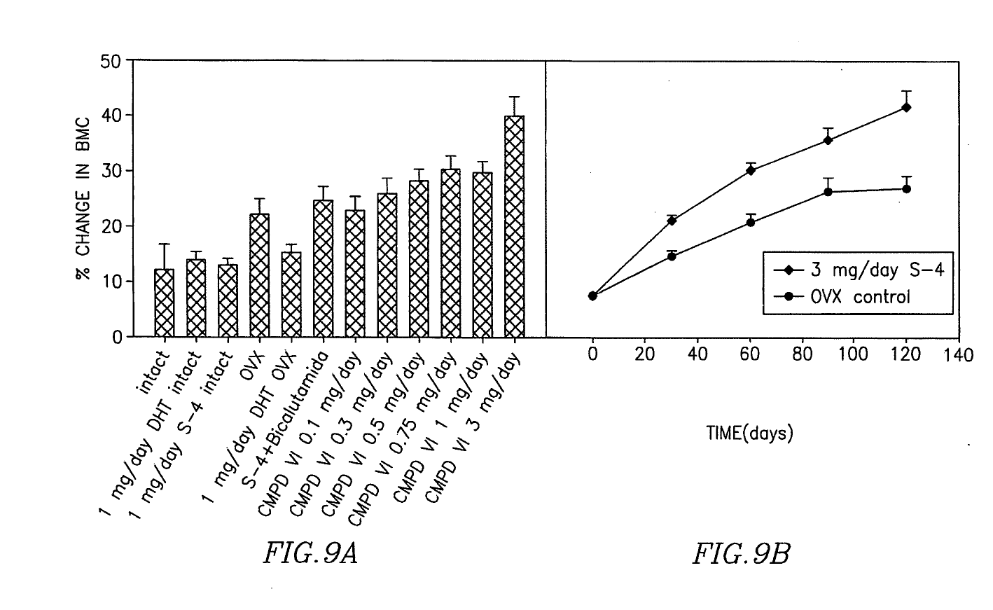

As shown in FIG. 9, Compound VI increased whole body BMC in a dose-dependent and time-dependent manner. The antiandrogen bicalutamide inhibited the effect of Compound VI in this model indicating drug effects were mediated through the androgen receptor. DHT was less potent than Compound VI in this model.

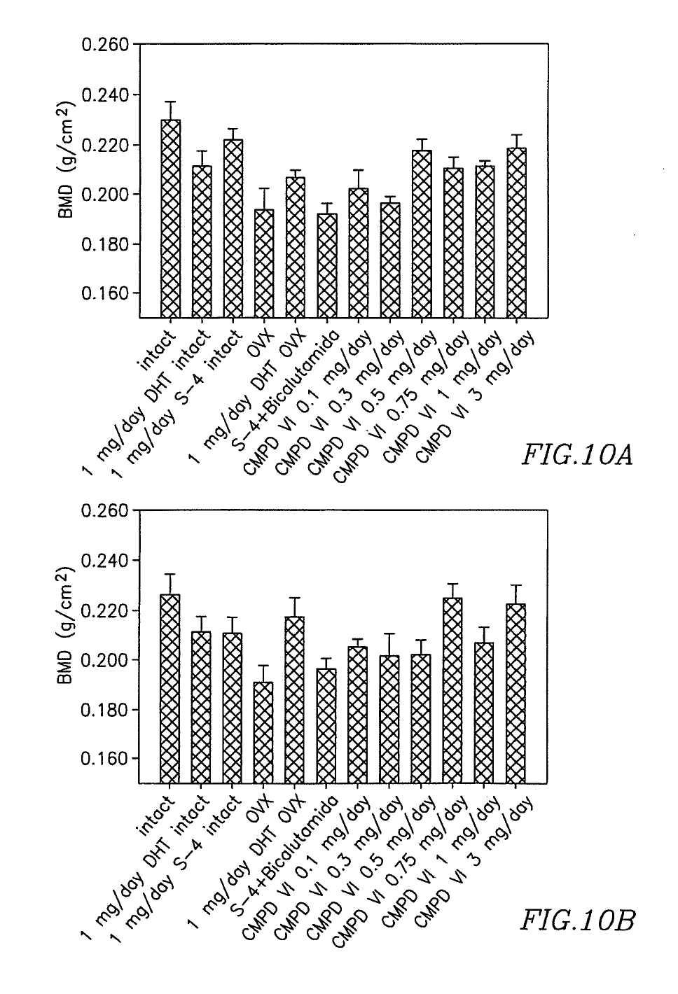

As shown in FIG. 10, Compound VI exerted a protective effect at both the L2-L4 vertebra and proximal femur. Bicalutamide abrogated the protective effect of Compund VI at both sites.

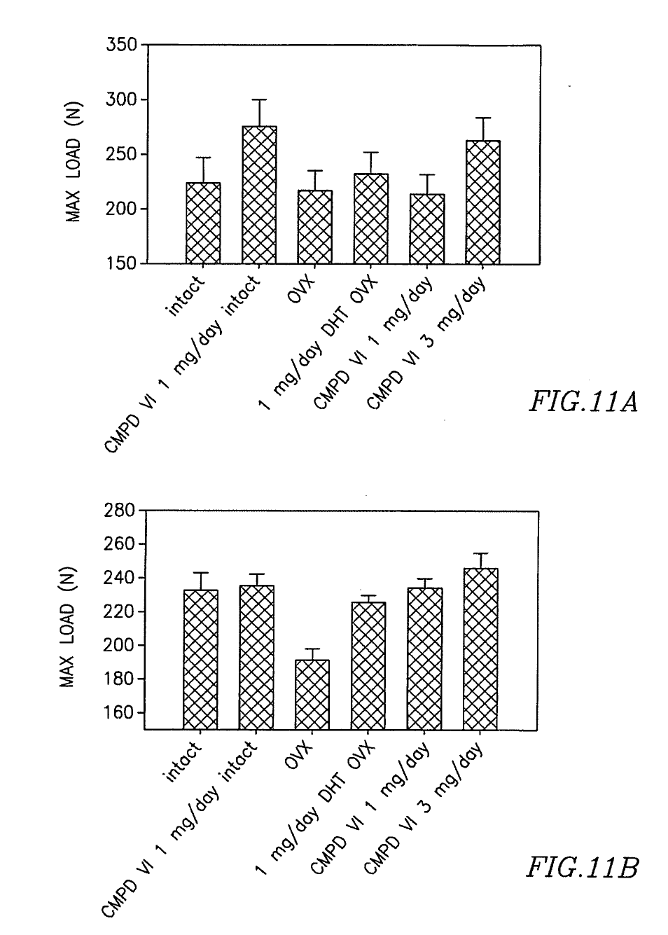

Biomechanical Strength: As shown in FIG. 11, Compound VI increased biomechanical strength of the L5 vertebra and femur, demonstrating that Compound VI has beneficial effects on trabecular and cortical bone.

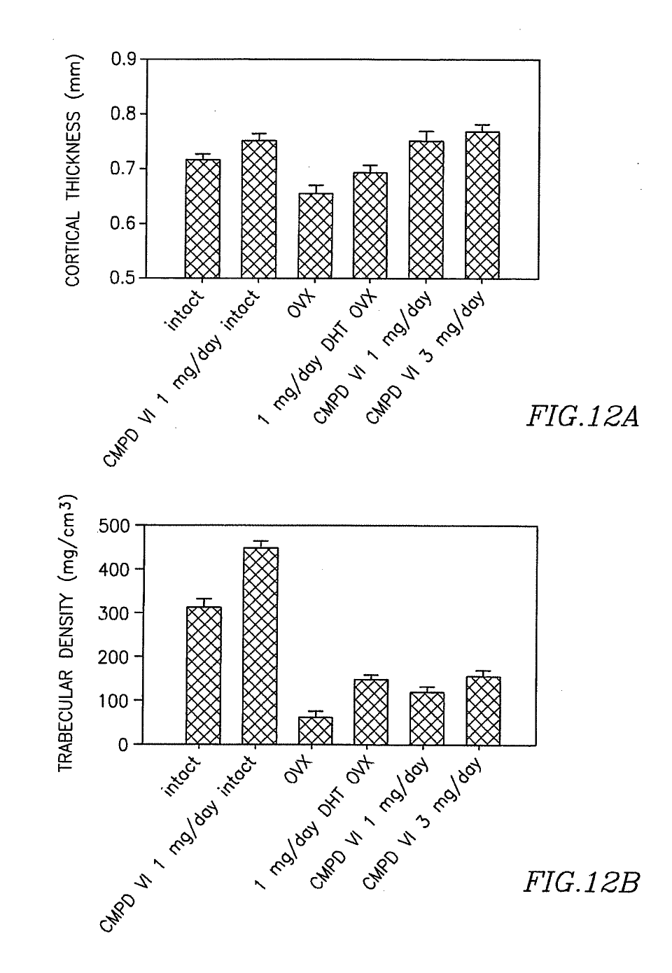

As shown in FIG. 12, Compound VI increased cortical thickness in the femoral mid-shaft, indicating anabolic action of Compound VI on cortical bone. In intact animals, Compound VI exerted a proliferative effect on trabecular density of the distal femur. However, in OVX animals Compound VI only partially prevented trabecular bone loss in the distal femur.

Compound VI exerts a protective effect on the skeleton following ovariectomy induced bone loss. Compound VI was more potent than DHT in this model and increased bone quality and biomechanical strength in both lumbar vertebrae and femur. Further, Compound VI also increased muscle mass in both gonadectomized and intact animals, which in combination with the protective skeletal effect should produce an additive benefit in reducing fracture rates in osteoporotic patients.

It will be appreciated by a person skilled in the art that the present invention is not limited by what has been particularly shown and described hereinabove. Rather, the scope of the invention is defined by the claims which follow:

1. A method of treating a male subject suffering from an Androgen Decline in Aging Male (ADAM)-associated condition, said method comprising the step of administering to said subject a selective androgen receptor modulator (SARM) compound.

2. The method of claim 1, comprising administering an analog, derivative, isomer, metabolite, pharmaceutically acceptable salt, pharmaceutical product, hydrate or N-oxide, prodrug, polymorph or crystal of said SARM compound, or any combination thereof.

3. The method according to claim 1, wherein said SARM compound is represented by the structure of formula I: 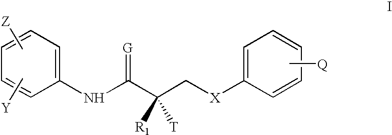
wherein
G is O or S;
X is a bond, O, CH₂, NH, Se, PR, NO or NR;
T is OH, OR, —NHCOCH₃, or NHCOR
Z is NO₂, CN, COOH, COR, NHCOR or CONHR;
Y is CF₃, F, I, Br, Cl, CN, CR₃ or SnR₃;
Q is alkyl, F, Cl, Br, I, CF₃, CN CR₃, SnR₃, NR₂, NHCOCH₃, NHCOCF₃, NHCOR, NHCONHR, NHCOOR, OCONHR, CONHR, NHCSCH₃, NHCSCF₃, NHCSR NHSO₂CH₃, NHSO₂R, OR, COR, OCOR, OSO₂R, SO₂R, SR, NCS, SCN, NCO, OCN; or Q together with the benzene ring to which it is attached is a fused ring system represented by structure A, B or C: 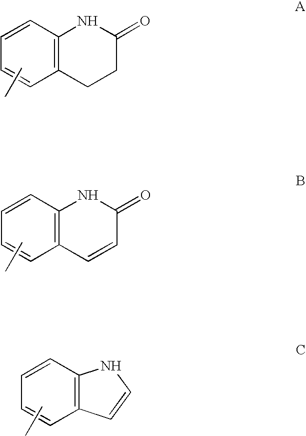
R is alkyl, haloalkyl, dihaloalkyl, trihaloalkyl, CH₂F, CHF₂, CF₃, CF₂CF₃, aryl, phenyl, F, Cl, Br, I, alkenyl or OH; and
R₁ is CH₃, CH₂F, CHF₂, CF₃, CH₂CH₃, or CF₂CF₃.

4. The method according to claim 1, wherein said SARM compound is represented by the structure of formula II. 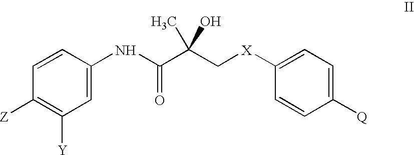
wherein
X is a bond, O, CH₂, NH, Se, PR, NO or NR;
Z is NO₂, CN, COOH, COR, NHCOR or CONHR;
Y is CF₃, F, I, Br, Cl, CN, CR₃ or SnR₃;
Q is alkyl, F, Cl, Br, I, CF₃, CN CR₃, SnR₃, NR₂, NHCOCH₃, NHCOCF₃, NHCOR, NHCONHR, NHCOOR, OCONHR, CONHR, NHCSCH₃, NHCSCF₃, NHCSR NHSO₂CH₃, NHSO₂R, OR, COR, OCOR, OSO₂R, SO₂R, SR, NCS, SCN, NCO, OCN; or Q together with the benzene ring to which it is attached is a fused ring system represented by structure A, B or C: 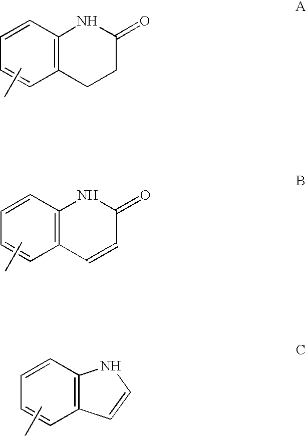
R is alkyl, haloalkyl, dihaloalkyl, trihaloalkyl, CH₂F, CHF₂, CF₃, CF₂CF₃, aryl, phenyl, F, Cl, Br, I, alkenyl or OH.

5. The method according to claim 1, wherein said SARM compound is represented by the structure of formula III. 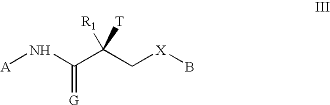
wherein
X is a bond, O, CH₂, NH, Se, PR, NO or NR;
G is O or S;
R₁ is CH₃, CH₂F, CHF₂, CF₃, CH₂CH₃, or CF₂CF₃;
T is OH, OR, —NHCOCH₃, or NHCOR;
R is alkyl, haloalkyl, dihaloalkyl, trihaloalkyl, CH₂F, CHF₂, CF₃, CF₂CF₃, aryl, phenyl, F, Cl, Br, I, alkenyl or OH;
A is a ring selected from: 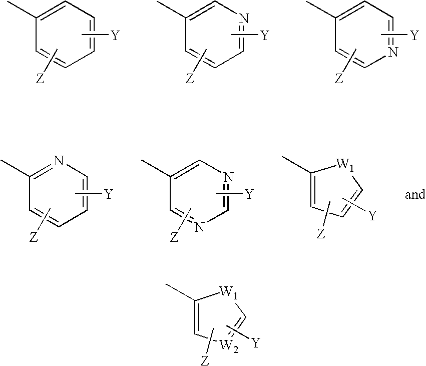
B is a ring selected from: 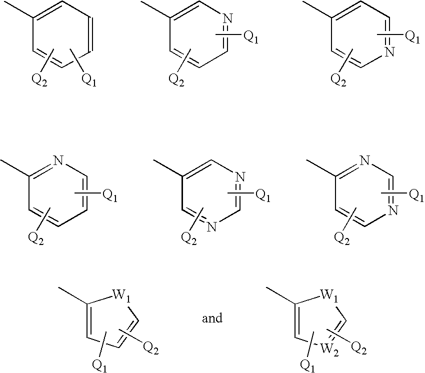
wherein
A and B cannot simultaneously be a benzene ring;
Z is NO₂, CN, COOH, COR, NHCOR or CONHR;
Y is CF₃, F, I, Br, Cl, CN CR₃ or SnR₃;
Q₁ and Q₂ are independently of each other a hydrogen, alkyl, F, Cl, Br, I, CF₃, CN CR₃, SnR₃, NR₂, NHCOCH₃, NHCOCF₃, NHCOR, NHCONHR, NHCOOR, OCONHR, CONHR, NHCSCH₃, NHCSCF₃, NHCSR NHSO₂CH₃, NHSO₂R, OR, COR, OCOR, OSO₂R, SO₂R, SR, NCS, SCN, NCO, OCN, 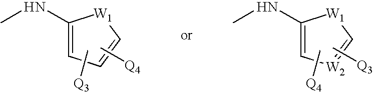
Q₃ and Q₄ are independently of each other a hydrogen, alkyl, F, Cl, Br, I, CF₃, CN CR₃, SnR₃, NR₂, NHCOCH₃, NHCOCF₃, NHCOR, NHCONHR, NHCOOR, OCONHR, CONHR, NHCSCH₃, NHCSCF₃, NHCSR NHSO₂CH₃, NHSO₂R,
OR, COR, OCOR, OSO₂R, SO₂R, SR, NCS, SCN, NCO or OCN;
W₁ is O, NH, NR, NO or S; and
W₂ is N or NO.

6. The method according to claim 1, wherein said SARM compound is represented by the structure of formula IV: 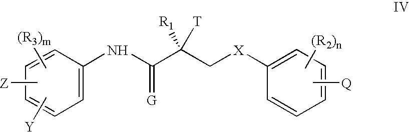
wherein
X is a bond, O, CH₂, NH, Se, PR, NO or NR;
G is O or S;
T is OH, OR, —NHCOCH₃, or NHCOR;
R is alkyl, haloalkyl, dihaloalkyl, trihaloalkyl, CH₂F, CHF₂, CF₃, CF₂CF₃, aryl, phenyl, F, Cl, Br, I, alkenyl or OH;
R₁ is CH₃, CH₂F, CHF₂, CF₃, CH₂CH₃, or CF₂CF₃;
R₂ is F, Cl, Br, I, CH₃, CF₃, OH, CN, NO₂, NHCOCH₃, NHCOCF₃, NHCOR, alkyl, arylalkyl, OR, NH₂, NHR, NR₂ or SR;
R₃ is F, Cl, Br, I, CN, NO₂, COR, COOH, CONHR, CF₃, SnR₃, or R₃ together with the benzene ring to which it is attached forms a fused ring system represented by the structure: 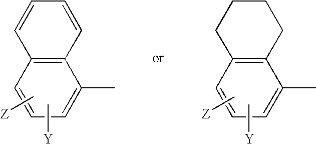
Z is NO₂, CN, COR, COOH, or CONHR;
Y is CF₃, F, Br, Cl, I, CN, or SnR₃;
Q is H, alkyl, F, Cl, Br, I, CF₃, CN CR₃, SnR₃, NR₂, NHCOCH₃, NHCOCF₃, NHCOR, NHCONHR, NHCOOR, OCONHR, CONHR, NHCSCH₃, NHCSCF₃, NHCSR NHSO₂CH₃, NHSO₂R, OH, OR, COR, OCOR, OSO₂R, SO₂R, SR, NCS, SCN, NCO, OCN; or Q together with the benzene ring to which it is attached is a fused ring system represented by structure A, B or C: 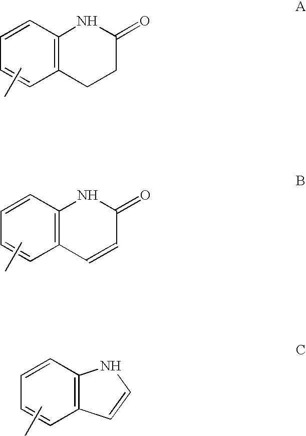
n is an integer of 1-4; and
m is an integer of 1-3.

7. The method according to claim 1, wherein said SARM compound is represented by the structure of formula V: 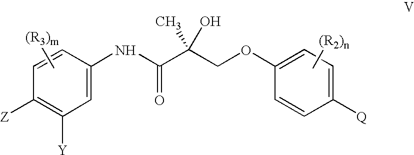
wherein
R₂ is F, Cl, Br, I, CH₃, CF₃, OH, CN, NO₂, NHCOCH₃, NHCOCF₃, NHCOR, alkyl, arylalkyl, OR, NH₂, NHR, NR₂ or SR;
R₃ is F, Cl, Br, I, CN, NO₂, COR, COOH, CONHR, CF₃, SnR₃, or R₃ together with the benzene ring to which it is attached forms a fused ring system represented by the structure: 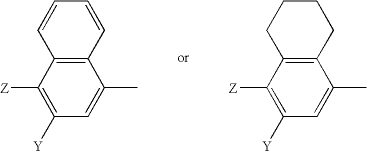
R is alkyl, haloalkyl, dihaloalkyl, trihaloalkyl, CH₂F, CHF₂, CF₃, CF₂CF₃, aryl, phenyl, F, Cl, Br, I, alkenyl or OH;
Z is NO₂, CN, COR, COOH, or CONHR;
Y is CF₃. F, Br, Cl, I, CN, or SnR₃;
Q is H, alkyl, F, Cl, Br, I, CF₃, CN CR₃, SnR₃, NR₂, NHCOCH₃, NHCOCF₃, NHCOR, NHCONHR, NHCOOR, OCONHR, CONHR, NHCSCH₃, NHCSCF₃, NHCSR NHSO₂CH₃, NHSO₂R, OH, OR, COR, OCOR, OSO₂R, SO₂R, SR, NCS, SCN, NCO, OCN; or Q together with the benzene ring to which it is attached is a fused ring system represented by structure A, B or C: 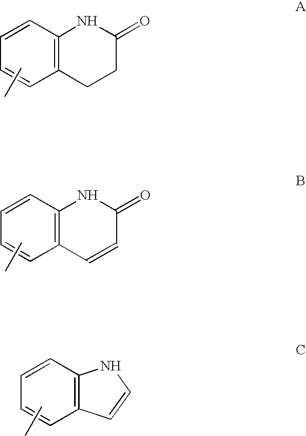
n is an integer of 1-4; and
m is an integer of 1-3.

8. The method according to claim 1, wherein said SARM compound is represented by the structure of formula VI.

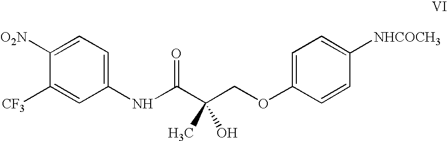

9. The method according to claim 1, wherein said SARM compound is represented by the structure of formula VII.

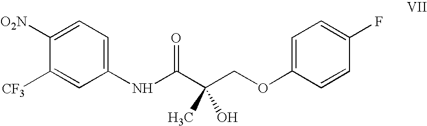

10. The method of claim 1, wherein the SARM is an androgen receptor agonist.

11. The method of claim 1, wherein the SARM is an androgen receptor antagonist.

12. The method of claim 1, wherein said SARM has an agonistic effect muscle or bone.

13. The method of claim 12, wherein said SARM has no effect or an antagonistic effect on prostate.

14. The method of claim 1, wherein said SARM has no effect on muscle or bone.

15. The method of claim 14, wherein said SARM has no effect or an antagonistic effect on prostate.

16. The method of claim 1, wherein said SARM has no effect or an antagonistic effect on prostate.

17. The method of claim 1, wherein said SARM penetrates the central nervous system (CNS).

18. The method of claim 1, wherein said SARM does not penetrate the central nervous system (CNS).

19. The method according to claim 1, comprising administering a pharmaceutical preparation comprising said SARM and/or its analog, derivative, isomer, metabolite, pharmaceutically acceptable salt, pharmaceutical product, hydrate, N-oxide, prodrug, polymorph, crystal, or any combination thereof; and a pharmaceutically acceptable carrier.

20. The method according to claim 19, comprising intravenously, intraarterially, or intramuscularly injecting to said subject said pharmaceutical preparation in liquid form; subcutaneously implanting in said subject a pellet containing said pharmaceutical preparation; orally administering to said subject said pharmaceutical preparation in a liquid or solid form; or topically applying to the skin surface of said subject said pharmaceutical preparation.

21. The method according to claim 19 wherein said pharmaceutical preparation is a pellet, a tablet, a capsule, a solution, a suspension, an emulsion, an elixir, a gel, a cream, a suppository or a parenteral formulation.

22. The method of claim 1, wherein said ADAM-associated condition is sexual dysfunction, decreased sexual libido, erectile dysfunction, hypogonadism, sarcopenia, osteopenia, osteoporosis, osteoporosis, alterations in cognition and mood, depression, anemia, hair loss, obesity, benign prostate hyperplasia, prostate cancer, or any combination thereof.

23. The method of claim 1, wherein said male subject is an aging male subject.

24. A method of preventing, suppressing, inhibiting or reducing the incidence of an Androgen Decline in Aging Male (ADAM)-associated condition in a male subject, said method comprising the step of administering to said subject a selective androgen receptor modulator (SARM) compound.

25. The method of claim 24, comprising administering an analog, derivative, isomer, metabolite, pharmaceutically acceptable salt, pharmaceutical product, hydrate or N-oxide, prodrug, polymorph or crystal of said SARM compound, or any combination thereof.

26. The method according to claim 24, wherein said SARM compound is represented by the structure of formula I: 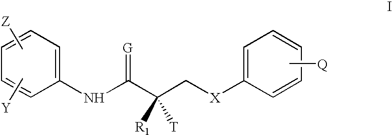
wherein
G is O or S;
X is a bond, O, CH₂, NH, Se, PR, NO or NR;
T is OH, OR, —NHCOCH₃, or NHCOR
Z is NO₂, CN, COOH, COR, NHCOR or CONHR;
Y is CF₃, F, I, Br, Cl, CN, CR₃ or SnR₃;
Q is alkyl, F, Cl, Br, I, CF₃, CN CR₃, SnR₃, NR₂, NHCOCH₃, NHCOCF₃, NHCOR, NHCONHR, NHCOOR, OCONHR, CONHR, NHCSCH₃, NHCSCF₃, NHCSR NHSO₂CH₃, NHSO₂R, OR, COR, OCOR, OSO₂R, SO₂R, SR, NCS, SCN, NCO, OCN; or Q together with the benzene ring to which it is attached is a fused ring system represented by structure A, B or C: 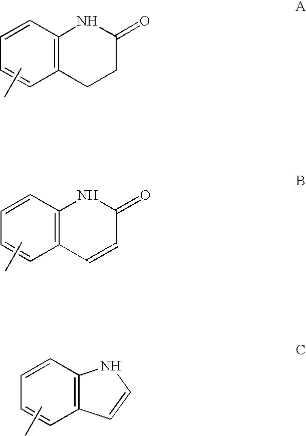
R is alkyl, haloalkyl, dihaloalkyl, trihaloalkyl, CH₂F, CHF₂, CF₃, CF₂CF₃, aryl, phenyl, F, Cl, Br, I, alkenyl or OH; and
R₁ is CH₃, CH₂F, CHF₂, CF₃, CH₂CH₃, or CF₂CF₃.

27. The method according to claim 24, wherein said SARM compound is represented by the structure of formula II. 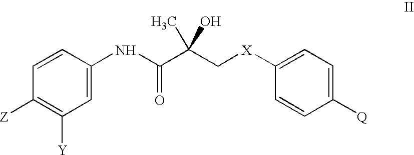
wherein
X is a bond, O, CH₂, NH, Se, PR, NO or NR;
Z is NO₂, CN, COOH, COR, NHCOR or CONHR;
Y is CF₃, F, I, Br, Cl, CN, CR₃ or SnR₃;
Q is alkyl, F, Cl, Br, I, CF₃, CN CR₃, SnR₃, NR₂, NHCOCH₃, NHCOCF₃, NHCOR, NHCONHR, NHCOOR, OCONHR, CONHR, NHCSCH₃, NHCSCF₃, NHCSR NHSO₂CH₃, NHSO₂R, OR, COR, OCOR, OSO₂R, SO₂R, SR, NCS, SCN, NCO, OCN; or Q together with the benzene ring to which it is attached is a fused ring system represented by structure A, B or C: 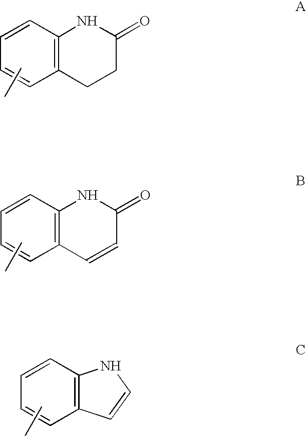
R is alkyl, haloalkyl, dihaloalkyl, trihaloalkyl, CH₂F, CHF₂, CF₃, CF₂CF₃, aryl, phenyl, F, Cl, Br, I, alkenyl or OH.

28. The method according to claim 24, wherein said SARM compound is represented by the structure of formula III. 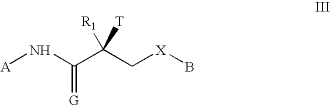
wherein
X is a bond, O, CH₂, NH, Se, PR, NO or NR;
G is O or S;
R₁ is CH₃, CH₂F, CHF₂, CF₃, CH₂CH₃, or CF₂CF₃;
T is OH, OR, —NHCOCH₃, or NHCOR;
R is alkyl, haloalkyl, dihaloalkyl, trihaloalkyl, CH₂F, CHF₂, CF₃, CF₂CF₃, aryl, phenyl, F, Cl, Br, I, alkenyl or OH;
A is a ring selected from: 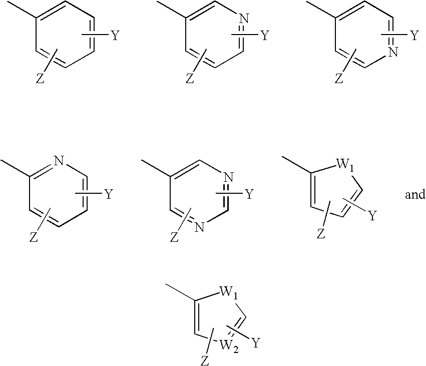
B is a ring selected from: 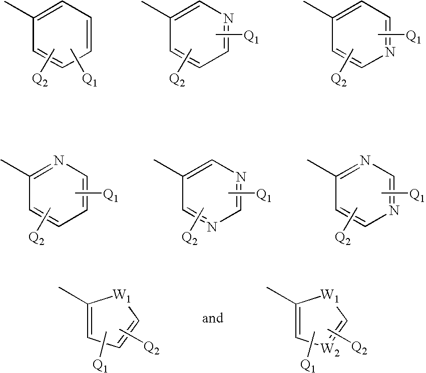
wherein
A and B cannot simultaneously be a benzene ring;
Z is NO₂, CN, COOH, COR, NHCOR or CONHR;
Y is CF₃, F, I, Br, Cl, CN CR₃ or SnR₃;
Q₁ and Q₂ are independently of each other a hydrogen, alkyl, F, Cl, Br, I, CF₃, CN CR₃, SnR₃, NR₂, NHCOCH₃, NHCOCF₃, NHCOR, NHCONHR, NHCOOR, OCONHR, CONHR, NHCSCH₃, NHCSCF₃, NHCSR NHSO₂CH₃, NHSO₂R, 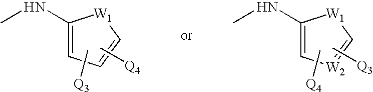
Q₃ and Q₄ are independently of each other a hydrogen, alkyl, F, Cl, Br, I, CF₃, CN CR₃, SnR₃, NR₂, NHCOCH₃, NHCOCF₃, NHCOR, NHCONHR, NHCOOR, OCONHR, CONHR, NHCSCH₃, NHCSCF₃, NHCSR NHSO₂CH₃, NHSO₂R, OR, COR, OCOR, OSO₂R, SO₂R, SR, NCS, SCN, NCO or OCN;
W₁ is O, NH, NR, NO or S; and
W₂ is N or NO.

29. The method according to claim 24, wherein said SARM compound is represented by the structure of formula IV: 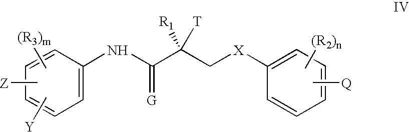
wherein
X is a bond, O, CH₂, NH, Se, PR, NO or NR;
G is O or S;
T is OH, OR, —NHCOCH₃, or NHCOR;
R is alkyl, haloalkyl, dihaloalkyl, trihaloalkyl, CH₂F, CHF₂, CF₃, CF₂CF₃, aryl, phenyl, F, Cl, Br, I, alkenyl or OH;
R₁ is CH₃, CH₂F, CHF₂, CF₃, CH₂CH₃, or CF₂CF₃;
R₂ is F, Cl, Br, I, CH₃, CF₃, OH, CN, NO₂, NHCOCH₃, NHCOCF₃, NHCOR, alkyl, arylalkyl, OR, NH₂, NHR, NR₂ or SR;
R₃ is F, Cl, Br, I, CN, NO₂, COR, COOH, CONHR, CF₃, SnR₃, or R₃ together with the benzene ring to which it is attached forms a fused ring system represented by the structure: 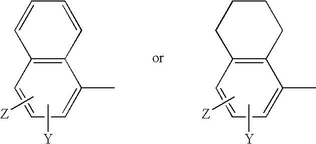
Z is NO₂, CN, COR, COOH, or CONHR;
Y is CF₃, F, Br, Cl, I, CN, or SnR₃;
Q is H, alkyl, F, Cl, Br, I, CF₃, CN CR₃, SnR₃, NR₂, NHCOCH₃, NHCOCF₃, NHCOR, NHCONHR, NHCOOR, OCONHR, CONHR, NHCSCH₃, NHCSCF₃, NHCSR NHSO₂CH₃, NHSO₂R, OH, OR, COR, OCOR, OSO₂R, SO₂R, SR, NCS, SCN, NCO, OCN; or Q together with the benzene ring to which it is attached is a fused ring system represented by structure A, B or C: 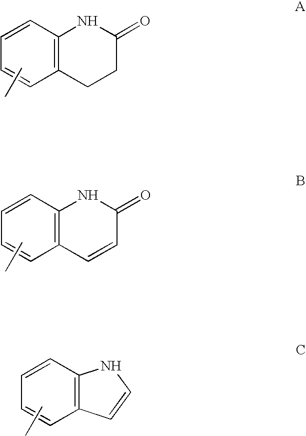
n is an integer of 1-4; and
m is an integer of 1-3.

30. The method according to claim 24, wherein said SARM compound is represented by the structure of formula V: 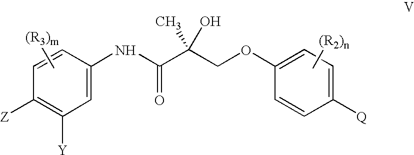
wherein
R₂ is F, Cl, Br, I, CH₃, CF₃, OH, CN, NO₂, NHCOCH₃, NHCOCF₃, NHCOR, alkyl, arylalkyl, OR, NH₂, NHR, NR₂ or SR;
R₃ is F, Cl, Br, I, CN, NO₂, COR, COOH, CONHR, CF₃, SnR₃, or R₃ together with the benzene ring to which it is attached forms a fused ring system represented by the structure: 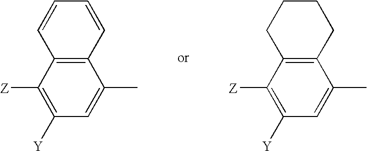
R is alkyl, haloalkyl, dihaloalkyl, trihaloalkyl, CH₂F, CHF₂, CF₃, CF₂CF₃, aryl, phenyl, F, Cl, Br, I, alkenyl or OH;
Z is NO₂, CN, COR, COOH, or CONHR;
Y is CF₃, F, Br, Cl, I, CN, or SnR₃;
Q is H, alkyl, F, Cl, Br, I, CF₃, CN CR₃, SnR₃, NR₂, NHCOCH₃, NHCOCF₃, NHCOR, NHCONHR, NHCOOR, OCONHR, CONHR, NHCSCH₃, NHCSCF₃, NHCSR NHSO₂CH₃, NHSO₂R, OH, OR, COR, OCOR, OSO₂R, SO₂R, SR, NCS, SCN, NCO, OCN; or Q together with the benzene ring to which it is attached is a fused ring system represented by structure A, B or C: 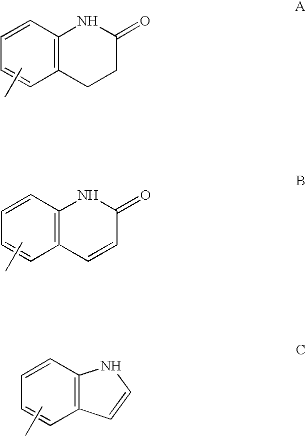
n is an integer of 1-4; and
m is an integer of 1-3.

31. The method according to claim 24, wherein said SARM compound is represented by the structure of formula VI.

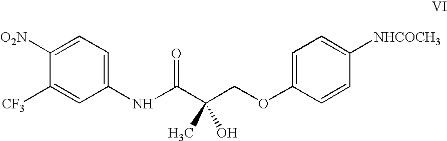

32. The method according to claim 24, wherein said SARM compound is represented by the structure of formula VII.

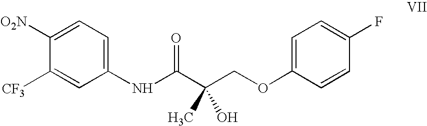

33. The method of claim 24, wherein the SARM is an androgen receptor agonist.

34. The method of claim 24, wherein the SARM is an androgen receptor antagonist.

35. The method of claim 24, wherein said SARM has an agonistic effect muscle or bone.

36. The method of claim 34, wherein said SARM has no effect or an antagonistic effect on prostate.

37. The method of claim 24, wherein said SARM has no effect on muscle or bone.

38. The method of claim 37, wherein said SARM has no effect or an antagonistic effect on prostate.

39. The method of claim 24, wherein said SARM has no effect or an antagonistic effect on prostate.

40. The method of claim 24, wherein said SARM penetrates the central nervous system (CNS).

41. The method of claim 24, wherein said SARM does not penetrate the central nervous system (CNS).

42. The method according to claim 24, comprising administering a pharmaceutical preparation comprising said SARM and/or its analog, derivative, isomer, metabolite, pharmaceutically acceptable salt, pharmaceutical product, hydrate, N-oxide, prodrug, polymorph, crystal, or any combination thereof, and a pharmaceutically acceptable carrier.

43. The method according to claim 42, comprising intravenously, intraarterially, or intramuscularly injecting to said subject said pharmaceutical preparation in liquid form; subcutaneously implanting in said subject a pellet containing said pharmaceutical preparation; orally administering to said subject said pharmaceutical preparation in a liquid or solid form; or topically applying to the skin surface of said subject said pharmaceutical preparation.

44. The method according to claim 42 wherein said pharmaceutical preparation is a pellet, a tablet, a capsule, a solution, a suspension, an emulsion, an elixir, a gel, a cream, a suppository or a parenteral formulation.

45. The method of claim 24, wherein said ADAM-associated condition is sexual dysfunction, decreased sexual libido, erectile dysfunction, hypogonadism, sarcopenia, osteopenia, osteoporosis, osteoporosis, alterations in cognition and mood, depression, anemia, hair loss, obesity, benign prostate hyperplasia, prostate cancer, or any combination thereof.

46. The method of claim 24, wherein said male subject is an aging male subject.

47. A method of treating a male subject suffering from is sexual dysfunction, decreased sexual libido, erectile dysfunction, hypogonadism, sarcopenia, osteopenia, osteoporosis, osteoporosis, alterations in cognition and mood, depression, anemia, hair loss, obesity, benign prostate hyperplasia or prostate cancer due to Androgen Decline in an Aging Male (ADAM), said method comprising the step of administering to said subject a selective androgen receptor modulator (SARM) compound.

48. The method of claim 47, comprising administering an analog, derivative, isomer, metabolite, pharmaceutically acceptable salt, pharmaceutical product, hydrate or N-oxide, prodrug, polymorph or crystal of said SARM compound, or any combination thereof.

49. The method according to claim 47, wherein said SARM compound is represented by the structure of formula I: 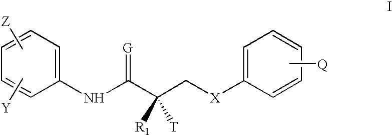
wherein
G is O or S;
X is a bond, O, CH₂, NH, Se, PR, NO or NR;
T is OH, OR, —NHCOCH₃, or NHCOR
Z is NO₂, CN, COOH, COR, NHCOR or CONHR;
Y is CF₃, F, I, Br, Cl, CN, CR₃ or SnR₃;
Q is alkyl, F, Cl, Br, I, CF₃, CN CR₃, SnR₃, NR₂, NHCOCH₃, NHCOCF₃, NHCOR, NHCONHR, NHCOOR, OCONHR, CONHR, NHCSCH₃, NHCSCF₃, NHCSR NHSO₂CH₃, NHSO₂R, OR, COR, OCOR, OSO₂R, SO₂R, SR, NCS, SCN, NCO, OCN; or Q together with the benzene ring to which it is attached is a fused ring system represented by structure A, B or C: 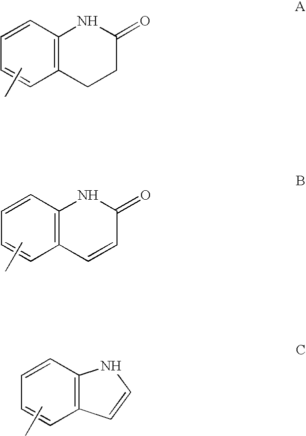
R is alkyl, haloalkyl, dihaloalkyl, trihaloalkyl, CH₂F, CHF₂, CF₃, CF₂CF₃, aryl, phenyl, F, Cl, Br, I, alkenyl or OH; and
R₁ is CH₃, CH₂F, CHF₂, CF₃, CH₂CH₃, or CF₂CF₃.

50. The method according to claim 47, wherein said SARM compound is represented by the structure of formula II. 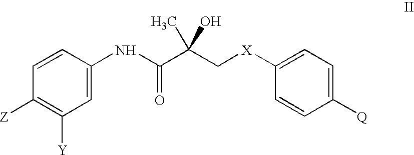
wherein
X is a bond, O, CH₂, NH, Se, PR, NO or NR;
Z is NO₂, CN, COOH, COR, NHCOR or CONHR;
Y is CF₃, F, I, Br, Cl, CN, CR₃ or SnR₃;
Q is alkyl, F, Cl, Br, I, CF₃, CN CR₃, SnR₃, NR₂, NHCOCH₃, NHCOCF₃, NHCOR, NHCONHR, NHCOOR, OCONHR, CONHR, NHCSCH₃, NHCSCF₃, NHCSR NHSO₂CH₃, NHSO₂R, OR, COR, OCOR, OSO₂R, SO₂R, SR, NCS, SCN, NCO, OCN; or Q together with the benzene ring to which it is attached is a fused ring system represented by structure A, B or C: 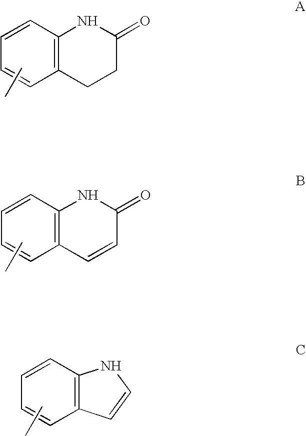
R is alkyl, haloalkyl, dihaloalkyl, trihaloalkyl, CH₂F, CHF₂, CF₃, CF₂CF₃, aryl, phenyl, F, Cl, Br, I, alkenyl or OH.

51. The method according to claim 47, wherein said SARM compound is represented by the structure of formula III. 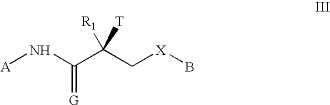
wherein
X is a bond, O, CH₂, NH, Se, PR, NO or NR;
G is O or S;
R₁ is CH₃, CH₂F, CHF₂, CF₃, CH₂CH₃, or CF₂CF₃;
T is OH, OR, —NHCOCH₃, or NHCOR;
R is alkyl, haloalkyl, dihaloalkyl, trihaloalkyl, CH₂F, CHF₂, CF₃, CF₂CF₃, aryl, phenyl, F, Cl, Br, I, alkenyl or OH;
A is a ring selected from: 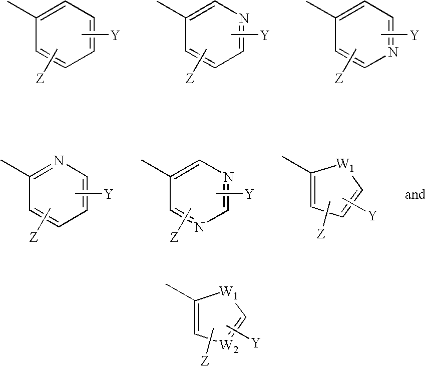
B is a ring selected from: 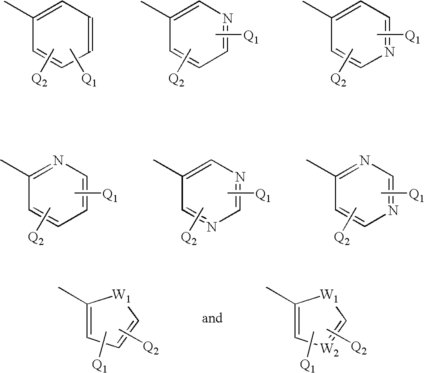
wherein
A and B cannot simultaneously be a benzene ring;
Z is NO₂, CN, COOH, COR, NHCOR or CONHR;
Y is CF₃, F, I, Br, Cl, CN CR₃ or SnR₃;
Q₁ and Q₂ are independently of each other a hydrogen, alkyl, F, Cl, Br, I, CF₃, CN CR₃, SnR₃, NR₂, NHCOCH₃, NHCOCF₃, NHCOR, NHCONHR, NHCOOR, OCONHR, CONHR, NHCSCH₃, NHCSCF₃, NHCSR NHSO₂CH₃, NHSO₂R, OR, COR, OCOR, OSO₂R, SO₂R, SR, NCS, SCN, NCO, OCN, 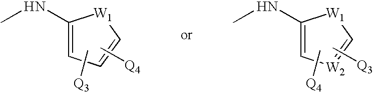
Q₃ and Q₄ are independently of each other a hydrogen, alkyl, F, Cl, Br, I, CF₃, CN CR₃, SnR₃, NR₂, NHCOCH₃, NHCOCF₃, NHCOR, NHCONHR, NHCOOR, OCONHR, CONHR, NHCSCH₃, NHCSCF₃, NHCSR NHSO₂CH₃, NHSO₂R, OR, COR, OCOR, OSO₂R, SO₂R, SR, NCS, SCN, NCO or OCN;
W₁ is O, NH, NR, NO or S; and
W₂ is N or NO.

52. The method according to claim 47, wherein said SARM compound is represented by the structure of formula IV: 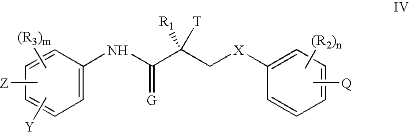
wherein
X is a bond, O, CH₂, NH, Se, PR, NO or NR;
G is O or S;
T is OH, OR, —NHCOCH₃, or NHCOR;
R is allyl, haloalkyl, dihaloalkyl, trihaloalkyl, CH₂F, CHF₂, CF₃, CF₂CF₃, aryl, phenyl, F, Cl, Br, I, alkenyl or OH;
R₁ is CH₃, CH₂F, CHF₂, CF₃, CH₂CH₃, or CF₂CF₃;
R₂ is F, Cl, Br, I, CH₃, CF₃, OH, CN, NO₂, NHCOCH₃, NHCOCF₃, NHCOR, alkyl, arylalkyl, OR, NH₂, NHR, NR₂ or SR;
R₃ is F, Cl, Br, I, CN, NO₂, COR, COOH, CONHR, CF₃, SnR₃, or R₃ together with the benzene ring to which it is attached forms a fused ring system represented by the structure: 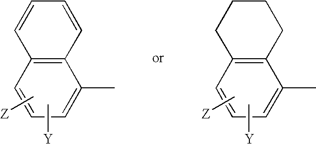
Z is NO₂, CN, COR, COOH, or CONHR;
Y is CF₃, F, Br, Cl, I, CN, or SnR₃;
Q is H, alkyl, F, Cl, Br, I, CF₃, CN CR₃, SnR₃, NR₂, NHCOCH₃, NHCOCF₃, NHCOR, NHCONHR, NHCOOR, OCONHR, CONHR, NHCSCH₃, NHCSCF₃, NHCSR NHSO₂CH₃, NHSO₂R, OH, OR, COR, OCOR, OSO₂R, SO₂R, SR, NCS, SCN, NCO, OCN; or Q together with the benzene ring to which it is attached is a fused ring system represented by structure A, B or C: 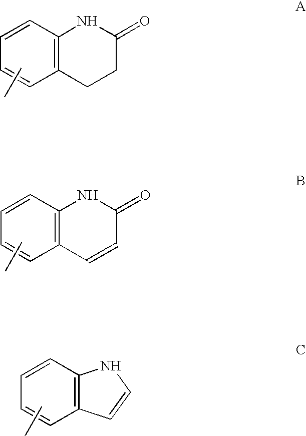
n is an integer of 1-4; and
m is an integer of 1-3.

53. The method according to claim 47, wherein said SARM compound is represented by the structure of formula V: 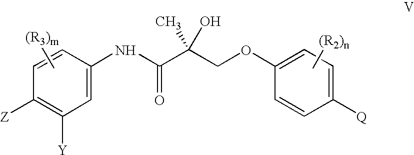
wherein
R₂ is F, Cl, Br, I, CH₃, CF₃, OH, CN, NO₂, NHCOCH₃, NHCOCF₃, NHCOR, alkyl, arylalkyl, OR, NH₂, NHR, NR₂ or SR;
R₃ is F, Cl, Br, I, CN, NO₂, COR, COOH, CONHR, CF₃, SnR₃, or R₃ together with the benzene ring to which it is attached forms a fused ring system represented by the structure: 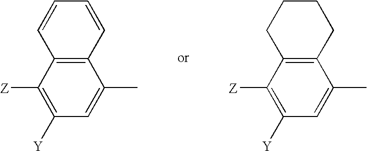
R is alkyl, haloalkyl, dihaloalkyl, trihaloalkyl, CH₂F, CHF₂, CF₃, CF₂CF₃, aryl, phenyl, F, Cl, Br, I, alkenyl or OH;
Z is NO₂, CN, COR, COOH, or CONHR;
Y is CF₃, F, Br, Cl, I, CN, or SnR₃;
Q is H, alkyl, F, Cl, Br, I, CF₃, CN CR₃, SnR₃, NR₂, NHCOCH₃, NHCOCF₃, NHCOR, NHCONHR, NHCOOR, OCONHR, CONHR, NHCSCH₃, NHCSCF₃, NHCSR NHSO₂CH₃, NHSO₂R, OH, OR, COR, OCOR, OSO₂R, SO₂R, SR, NCS, SCN, NCO, OCN; or Q together with the benzene ring to which it is attached is a fused ring system represented by structure A, B or C: 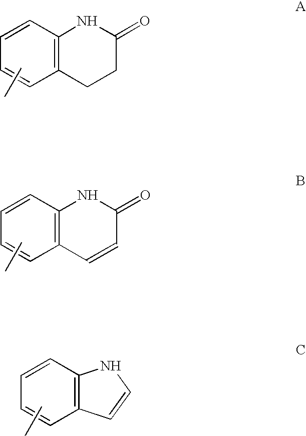
n is an integer of 1-4; and
m is an integer of 1-3.

54. The method according to claim 47, wherein said SARM compound is represented by the structure of formula VI.

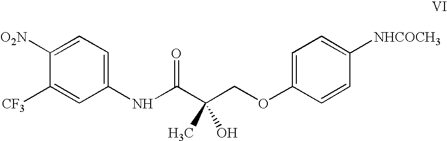

55. The method according to claim 47, wherein said SARM compound is represented by the structure of formula VII.

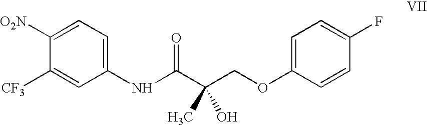

56. The method of claim 47, wherein the SARM is an androgen receptor agonist.

57. The method of claim 47, wherein the SARM is an androgen receptor antagonist.

58. The method of claim 47, wherein said SARM has an agonistic effect muscle or bone.

59. The method of claim 58, wherein said SARM has no effect or an antagonistic effect on prostate.

60. The method of claim 47, wherein said SARM has no effect on muscle or bone.

61. The method of claim 60, wherein said SARM has no effect or an antagonistic effect on prostate.

62. The method of claim 47, wherein said SARM has no effect or an antagonistic effect on prostate.

63. The method of claim 45, wherein said SARM penetrates the central nervous system (CNS).

64. The method of claim 47, wherein said SARM does not penetrate the central nervous system (CNS).

65. The method according to claim 47, comprising administering a pharmaceutical preparation comprising said SARM and/or its analog, derivative, isomer, metabolite, pharmaceutically acceptable salt, pharmaceutical product, hydrate, N-oxide, prodrug, polymorph, crystal, or any combination thereof; and a pharmaceutically acceptable carrier.

66. The method according to claim 65, comprising intravenously, intraarterially, or intramuscularly injecting to said subject said pharmaceutical preparation in liquid form; subcutaneously implanting in said subject a pellet containing said pharmaceutical preparation; orally administering to said subject said pharmaceutical preparation in a liquid or solid form; or topically applying to the skin surface of said subject said pharmaceutical preparation.

67. The method according to claim 65 wherein said pharmaceutical preparation is a pellet, a tablet, a capsule, a solution, a suspension, an emulsion, an elixir, a gel, a cream, a suppository or a parenteral formulation.

68. The method of claim 47, wherein said male subject is an aging male subject.

69. A method of preventing, suppressing, inhibiting or reducing the incidence of an Androgen Decline in an Aging Male (ADAM)-associated condition selected from sexual dysfunction, decreased sexual libido, erectile dysfunction, hypogonadism, sarcopenia, osteopenia, osteoporosis, osteoporosis, alterations in cognition and mood, depression, anemia, hair loss, obesity, benign prostate hyperplasia and prostate cancer in a male subject, said method comprising the step of administering to said subject a selective androgen receptor modulator (SARM) compound.

70. The method of claim 69, comprising administering an analog, derivative, isomer, metabolite, pharmaceutically acceptable salt, pharmaceutical product, hydrate or N-oxide, prodrug, polymorph or crystal of said SARM compound, or any combination thereof.

71. The method according to claim 69, wherein said SARM compound is represented by the structure of formula I: 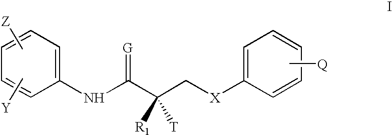
wherein
G is O or S;
X is a bond, O, CH₂, NH, Se, PR, NO or NR;
T is OH, OR, —NHCOCH₃, or NHCOR
Z is NO₂, CN, COOH, COR, NHCOR or CONHR;
Y is CF₃, F, I, Br, Cl, CN, CR₃ or SnR₃;
Q is alkyl, F, Cl, Br, I, CF₃, CN CR₃, SnR₃, NR₂, NHCOCH₃, NHCOCF₃, NHCOR, NHCONHR, NHCOOR, OCONHR, CONHR, NHCSCH₃, NHCSCF₃, NHCSR NHSO₂CH₃, NHSO₂R, OR, COR, OCOR, OSO₂R, SO₂R, SR, NCS, SCN, NCO, OCN; or Q together with the benzene ring to which it is attached is a fused ring system represented by structure A, B or C: 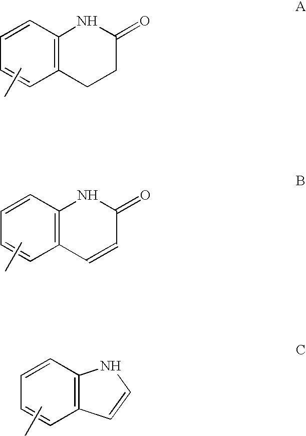
R is alkyl, haloalkyl, dihaloalkyl, trihaloalkyl, CH₂F, CHF₂, CF₃, CF₂CF₃, aryl, phenyl, F, Cl, Br, I, alkenyl or OH; and
R₁ is CH₃, CH₂F, CHF₂, CF₃, CH₂CH₃, or CF₂CF₃.

72. The method according to claim 69, wherein said SARM compound is represented by the structure of formula II. 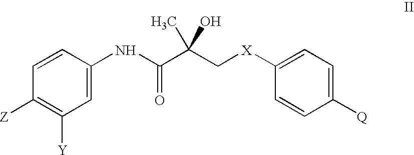
wherein
X is a bond, O, CH₂, NH, Se, PR, NO or NR;
Z is NO₂, CN, COOH, COR, NHCOR or CONHR;
Y is CF₃, F, I, Br, Cl, CN, CR₃ or SnR₃;
Q is alkyl, F, Cl, Br, I, CF₃, CN CR₃, SnR₃, NR₂, NHCOCH₃, NHCOCF₃, NHCOR, NHCONHR, NHCOOR, OCONHR, CONHR, NHCSCH₃, NHCSCF₃, NHCSR NHSO₂CH₃, NHSO₂R, OR, COR, OCOR, OSO₂R, SO₂R, SR, NCS, SCN, NCO, OCN; or Q together with the benzene ring to which it is attached is a fused ring system represented by structure A, B or C: 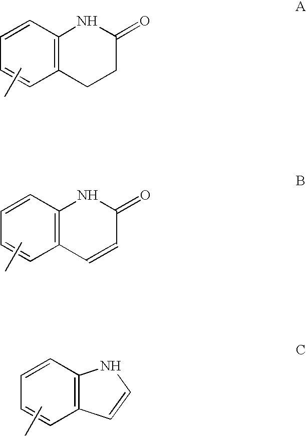
R is alkyl, haloalkyl, dihaloalkyl, trihaloalkyl, CH₂F, CHF₂, CF₃, CF₂CF₃, aryl, phenyl, F, Cl, Br, I, alkenyl or OH.

73. The method according to claim 69, wherein said SARM compound is represented by the structure of formula III. 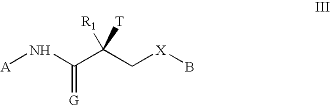
wherein
X is a bond, O, CH₂, NH, Se, PR, NO or NR;
G is O or S;
R₁ is CH₃, CH₂F, CHF₂, CF₃, CH₂CH₃, or CF₂CF₃;
T is OH, OR, —NHCOCH₃, or NHCOR;
R is alkyl, haloalkyl, dihaloalkyl, trihaloalkyl, CH₂F, CHF₂, CF₃, CF₂CF₃, aryl, phenyl, F, Cl, Br, I, alkenyl or OH;
A is a ring selected from: 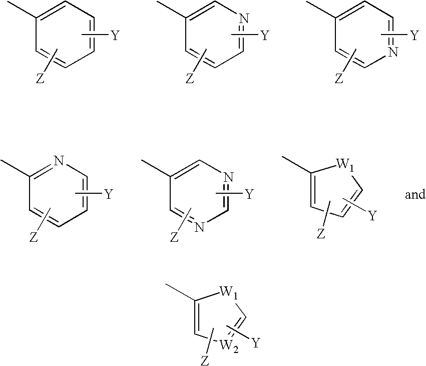
B is a ring selected from: 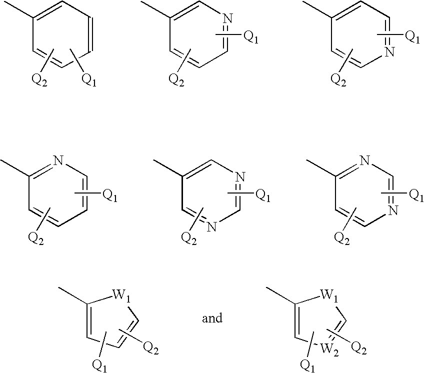
wherein
A and B cannot simultaneously be a benzene ring;
Z is NO₂, CN, COOH, COR, NHCOR or CONHR;
Y is CF₃, F, I, Br, Cl, CN CR₃ or SnR₃;
Q₁ and Q₂ are independently of each other a hydrogen, alkyl, F, Cl, Br, I, CF₃, CN CR₃, SnR₃, NR₂, NHCOCH₃, NHCOCF₃, NHCOR, NHCONHR, NHCOOR, OCONHR, CONHR, NHCSCH₃, NHCSCF₃, NHCSR NHSO₂CH₃, NHSO₂R, OR, COR, OCOR, OSO₂R, SO₂R, SR, NCS, SCN, NCO, OCN, 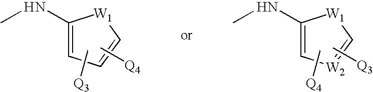
Q₃ and Q₄ are independently of each other a hydrogen, alkyl, F, Cl, Br, I, CF₃, CN CR₃, SnR₃, NR₂, NHCOCH₃, NHCOCF₃, NHCOR, NHCONHR, NHCOOR, OCONHR, CONHR, NHCSCH₃, NHCSCF₃, NHCSR NHSO₂CH₃, NHSO₂R, OR, COR, OCOR, OSO₂R, SO₂R, SR, NCS, SCN, NCO or OCN;
W₁ is O, NH, NR, NO or S; and
W₂ is N or NO.

74. The method according to claim 69, wherein said SARM compound is represented by the structure of formula IV: 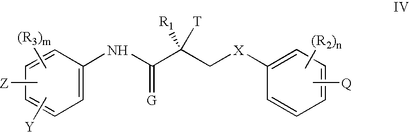
wherein
X is a bond, O, CH₂, NH, Se, PR, NO or NR;
G is O or S;
T is OH, OR, —NHCOCH₃, or NHCOR;
R is alkyl, haloalkyl, dihaloalkyl, trihaloalkyl, CH₂F, CHF₂, CF₃, CF₂CF₃, aryl, phenyl, F, Cl, Br, I, alkenyl or OH;
R₁ is CH₃, CH₂F, CHF₂, CF₃, CH₂CH₃, or CF₂CF₃;
R₂ is F, Cl, Br, I, CH₃, CF₃, OH, CN, NO₂, NHCOCH₃, NHCOCF₃, NHCOR, alkyl, arylalkyl, OR, NH₂, NHR, NR₂ or SR;
R₃ is F, Cl, Br, I, CN, NO₂, COR, COOH, CONHR, CF₃, SnR₃, or R₃ together with the benzene ring to which it is attached forms a fused ring system represented by the structure: 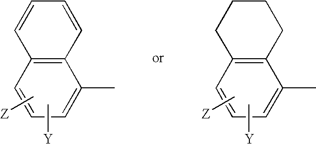
Z is NO₂, CN, COR, COOH, or CONHR;
Y is CF₃, F, Br, Cl, I, CN, or SnR₃;
Q is H, alkyl, F, Cl, Br, I, CF₃, CN CR₃, SnR₃, NR₂, NHCOCH₃, NHCOCF₃, NHCOR, NHCONHR, NHCOOR, OCONHR, CONHR, NHCSCH₃, NHCSCF₃, NHCSR NHSO₂CH₃, NHSO₂R, OH, OR, COR, OCOR, OSO₂R, SO₂R, SR, NCS, SCN, NCO, OCN; or Q together with the benzene ring to which it is attached is a fused ring system represented by structure A, B or C: 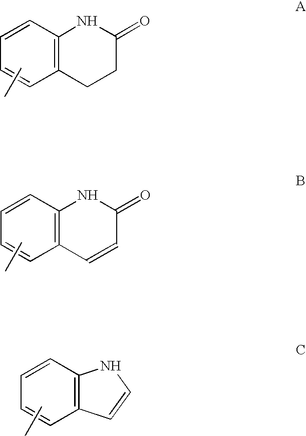
n is an integer of 1-4; and
m is an integer of 1-3.

75. The method according to claim 69, wherein said SARM compound is represented by the structure of formula V: 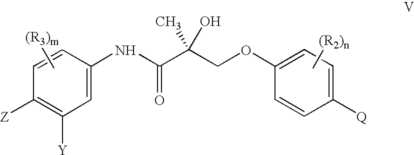
wherein
R₂ is F, Cl, Br, I, CH₃, CF₃, OH, CN, NO₂, NHCOCH₃, NHCOCF₃, NHCOR, alkyl, arylalkyl, OR, NH₂, NHR, NR₂ or SR;
R₃ is F, Cl, Br, I, CN, NO₂, COR, COOH, CONHR, CF₃, SnR₃, or R₃ together with the benzene ring to which it is attached forms a fused ring system represented by the structure: 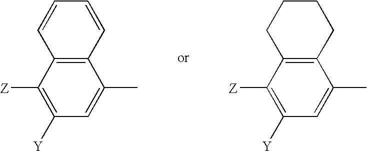
R is alkyl, haloalkyl, dihaloalkyl, trihaloalkyl, CH₂F, CHF₂, CF₃, CF₂CF₃, aryl, phenyl, F, Cl, Br, I, alkenyl or OH;
Z is NO₂, CN, COR, COOH, or CONHR;
Y is CF₃, F, Br, Cl, I, CN, or SnR₃;
Q is H, alkyl, F, Cl, Br, I, CF₃, CN CR₃, SnR₃, NR₂, NHCOCH₃, NHCOCF₃, NHCOR, NHCONHR, NHCOOR, OCONHR, CONHR, NHCSCH₃, NHCSCF₃, NHCSR NHSO₂CH₃, NHSO₂R, OH, OR, COR, OCOR, OSO₂R, SO₂R, SR, NCS, SCN, NCO, OCN; or Q together with the benzene ring to which it is attached is a fused ring system represented by structure A, B or C: 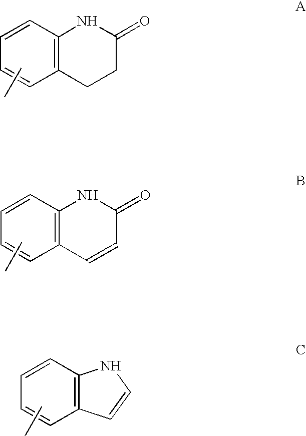
n is an integer of 1-4; and
m is an integer of 1-3.

76. The method according to claim 69, wherein said SARM compound is represented by the structure of formula VI.

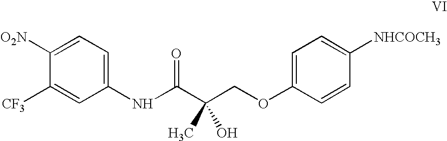

77. The method according to claim 69, wherein said SARM compound is represented by the structure of formula VII.

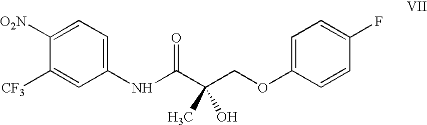

78. The method of claim 69, wherein the SARM is an androgen receptor agonist.

79. The method of claim 69, wherein the SARM is an androgen receptor antagonist.

80. The method of claim 69, wherein said SARM has an agonistic effect muscle or bone.

81. The method of claim 80, wherein said SARM has no effect or an antagonistic effect on prostate.

82. The method of claim 69, wherein said SARM has no effect on muscle or bone.

83. The method of claim 82, wherein said SARM has no effect or an antagonistic effect on prostate.

84. The method of claim 69, wherein said SARM has no effect or an antagonistic effect on prostate.

85. The method of claim 69, wherein said SARM penetrates the central nervous system (CNS).

86. The method of claim 69, wherein said SARM does not penetrate the central nervous system (CNS).

87. The method according to claim 69, comprising administering a pharmaceutical preparation comprising said SARM and/or its analog, derivative, isomer, metabolite, pharmaceutically acceptable salt, pharmaceutical product, hydrate, N-oxide, prodrug, polymorph, crystal, or any combination thereof; and a pharmaceutically acceptable carrier.

88. The method according to claim 87, comprising intravenously, intraarterially, or intramuscularly injecting to said subject said pharmaceutical preparation in liquid form; subcutaneously implanting in said subject a pellet containing said pharmaceutical preparation; orally administering to said subject said pharmaceutical preparation in a liquid or solid form; or topically applying to the skin surface of said subject said pharmaceutical preparation.

89. The method according to claim 87, wherein said pharmaceutical preparation is a pellet, a tablet, a capsule, a solution, a suspension, an emulsion, an elixir, a gel, a cream, a suppository or a parenteral formulation.

90. The method of claim 69, wherein said male subject is an aging male subject.
